(19) United States
(12) Patent Application Publication
(10) Pub. No.: US 2026/0102767 A1
(43) Pub. Date: Apr. 16, 2026

(54) SYSTEMS, DEVICES AND METHODS FOR RECOVERING CELLS AND CELLULAR COMPONENTS

(71) Applicant: Corramedical, Inc., Lakeside, CA (US)

(72) Inventors: Wilfrido MOJICA, Amherst, NY (US); Alexander ARROW, Lakeside, CA (US)

(21) Appl. No.: 19/338,736

(22) Filed: Sep. 24, 2025

Related U.S. Application Data
(60) Provisional application No. 63/707,908, filed on Oct. 16, 2024.

(51) Int. Cl. B01L 3/00; B01L 9/00

(52) U.S. Cl. CPC B01L 3/50255; B01L 9/00; B01L 2300/047; B01L 2300/0681; B01L 2300/0864; B01L 2400/0478

(57) ABSTRACT

Devices and methods for recovering cells and cellular components are disclosed herein. In an embodiment, a device for recovering cells and cellular components includes a plurality of collection receptacles. The plurality of collection receptacles are positioned and arranged with respect to each other so as to separate fluid with the loose cells and cellular fragments into separate aliquots. The plurality of collection receptacles are also separable from each other after the fluid with the loose cells and cellular fragments has been separated into the separate aliquots.

### PRIORITY

This application claims priority to U.S. Provisional Patent Application No. 63/707,908, filed Oct. 16, 2024, entitled “Systems and Methods for Recovering Cells and Cellular Components,” the entire disclosure of which is incorporated herein by reference and relied upon.

### BACKGROUND

### Technical Field

The present disclosure generally relates to systems, devices and methods for recovering cells and cellular components. More specifically, the present disclosure generally relates to systems, devices and methods for recovering cells and cellular components from excised solid tissue samples and making them available, for example, for cancer research in the fields of exosomes, metabolomics, transcriptomics, proteomics and epigenomics, and potentially for patient-specific diagnostic use in those same fields.

### Background Information

Tissue specimens can be resected and processed in different ways. For example, FIG. 1 illustrates how a primary bladder cancer tumor is resected and excised biopsy specimens are received after a procedure called a TransUrethral Resection of Bladder Tumor (TURBT), which is performed in patients with primary and recurrent urothelial cancers in the bladder. Image 1(A) shows how a resectoscope (a heated wire loop attached to a cystoscope) enters the bladder of a patient with a bladder tumor. Images 1(B), 1(C) and 1(D) show how the bladder tumor is visualized and cut. Image 1(E) illustrates the general appearance of solid tumor specimens as they arrive in the clinical pathology lab of a hospital. As shown in image 1(E), multiple irregularly shaped fragments of tissue are sent to the pathology lab, and each of them is typically friable and therefore crumbling into smaller pieces.

FIG. 2 illustrates how pathology laboratories typically prepares specimens for a process called formalin-fixation, paraffin-embedding (FFPE). This method, which has been the standard since the 1960s, preserves tissue for histopathologic evaluations under a brightfield microscope. Image 2(A) shows the fragments of the tumor retrieved by the cystoscope. Image 2(B) shows the standard biopsy bag, while image 2(C) shows a standard plastic tissue cassette. Image 2(D) shows the tumor fragments are placed in the biopsy bag within the plastic cassette. Image 2(E) shows how the tissue cassette baskets containing tissue specimens from different patients are often all placed together in a single processor box. The biopsy bags come in various sizes.

FIG. 3 shows three examples of standard biopsy bags. The typical pore size of these biopsy bags is 150 to 225 micrometers. Because of the friable nature of cells in certain types of tumors, and the pore size in these biopsy bags, tumor cells can seep out and be a source of contamination in other unrelated patient cases, for example, when placed in a processor box as shown in FIG. 2, which typically contains specimens from more than one patient. This has been reported in the pathology literature as a habitual source for contamination.

### SUMMARY

When performing an excisional biopsy procedure on a suspected tumor (a tissue mass) in a patient, the standard process for handling solid tissue specimens that are harvested wastes opportunities for additional diagnostic information. One reason is that the entire tissue specimen obtained is subject to the FFPE process. This results in the specimen's tissue quality becoming suboptimal for molecular analysis and completely incongruent with emerging technologies that hold the potential to improve upon patient diagnostics, such as the emerging “-omics” fields of metabolomics, transcriptomics, proteomics, and epigenomics. Promising technologies for cancer diagnostics involving the measurement of biomarker analytes such as exosomes, metabolites, and the growth of organoids are either associated with problems or impossible to employ when using FFPE-treated tissue as starting material. The reason the entire tissue specimen obtained is subject to the FFPE process—and no portion of it is held back to be available for molecular testing—is that pathologists who are charged with the task of finding any visual evidence of cancerous changes are loathe to give up any portion of the solid tissue for fear of missing the one part in which such cancerous changes may be evident via microscopy. The visual cancerous changes they are looking for, such as cells that are breaking free of their natural tethering to their basement membranes, or invading their neighboring tissue capsules, lamina, or organ sheaths, may be present broadly but may also be present only in a single part of a substantial tissue specimen.

Conversely, commercially derived cell lines, which are clones of cells originally sourced from a single tumor (from a patient who may have lived decades ago) and genetically modified to make them “immortal,” often used for investigational studies of exosomes and metabolomics, are not as representative of human tumor cells as cells derived from the tumor of the patient being treated. Additionally, obtaining access to cell-culture quality cells from a patient's tumor (what pathologists call “clinical material”) is often difficult because almost all tissue is processed by FFPE. Tissue processed by FFPE has been irrevocably altered by formalin (formaldehyde), which effectively wrecks it as a source of cell-culture quality cells and cellular components for the -omics. That system of tissue processing is heavily entrenched in the current clinical workflow.

The present disclosure provides a system and method that captures the full utility of clinical specimens, without requiring the pathologist to relinquish any of the solid tissue specimen for examination via microscopy. As a result, promising technologies to identify and study new biomarkers and cell behavior can be carried out on original, unbiased, human tissue-based specimens—potentially enabling tumors to be detected in an earlier time frame and specific mutations identified. That, in turn, may result in some patients'cancers being identified at an earlier, more treatable stage, which means that some patients whose only treatment options would have been radiation therapy and chemotherapy, along with their unavoidable side effects, can be eligible for more patient-friendly, tumor-deadly precision medicine therapies. The disclosed system and method are capable of recovering these analytes from tissue specimens without compromising or sacrificing the diagnostic value of the tissue for standard histopathologic evaluation. The disclosed method can also be performed at any pathology laboratory that obtains the system.

The present disclosure relates generally to container and filtration systems, and specifically to technologies for clinical pathology laboratory technicians and clinicians who perform tumor excision biopsy procedures and the pathologists who assist them to recover cells and cellular components from excised biopsy solid tissue samples that would otherwise go unutilized, in order to make them available for cancer-related diagnostic measurements in the fields of exosomes, metabolomics, transcriptomics, proteomics, epigenomics and organoid growth.

The present disclosure provides a specimen processing tool capable of providing a way to capture the opportunities for additional diagnostic information that are today being missed due to a lack of availability of formalin-free analytes and biomarkers from biopsy specimens. The tool is a simple yet effective device that takes advantage of the nature of tumor specimens commonly seen at the fresh, unfixed state. Its use also addresses and fixes a problem with methods in the pathology laboratory that presently do not entirely prevent specimen contamination. Combining these two principles allows for the maximal utilization of clinical material to create several aliquot specimens for multi-omic studies that heretofore were inaccessible due to barriers created by traditional FFPE specimen processing. The disclosed device is a convenient simple-to use device that recovers cells and cellular components currently being wasted from excised tissue samples collected in routine clinical practice and make them available for cancer research in the fields of exosomes and metabolomics, with potentially transcriptomics, proteomics, epigenomics and organoid growth as well.

In an embodiment, the systems and methods disclosed herein, when applied to bladder cancers resected by the TURBT method, create specimens for multi-omic examination by enabling the collection of diagnostic tumor cells that are undamaged by formalin and would otherwise be wasted.

In an embodiment, the systems and methods disclosed herein enable a user to create aliquots of specimens derived from the direct contact with these tumor cells. Aliquots consisting of exosomes, metabolites, viable cells for organoid growth, and cells unexposed to formalin fixation and therefore suitable for proteomic or molecular testing will be created—a library of specimens that can be used for multi-omic biomarker discovery or testing. The tissue in the biopsy bag and its corresponding cassette proceed to FFPE, so this approach does not compromise current tissue processing protocols. A secondary clinical advantage of this approach will be reducing the risk for tumor tissue contamination between samples harvested from different patients.

In an embodiment, the systems and methods disclosed herein make the collection of multiple analytes from a single tissue source possible without the need for instrumentation and pipetting. It is intended to be used by biobanks seeking to create multiple specimens from a single tissue source. The creation and separation of these additional specimens from the original parent tissue, which moves on to FFPE processing, allows for biomarker assessment and assignment from those daughter aliquots to the parental phenotype.

In an embodiment, the systems and methods disclosed herein allow for cells that would normally be lost during processing (and become a source of contamination) to instead be captured in fluid and used as aliquots for exosomes, metabolomics, growth of organoids, proteomics and molecular studies. An enormous advantage of this approach over current biomarker studies using fluid specimens (e.g., urine), is that the fluid collected by this approach was in direct contact with the tumor cells, and therefore far more representative of tumor-specific biomarkers than fluid collected from urine, which by its nature contains biomarkers from all over the body—only a tiny fraction of which would originate from tumor cells.

A first aspect of the present disclosure is to provide a device for recovering cells and cellular components. The device includes a plurality of collection receptacles. The plurality of collection receptacles are positioned and arranged with respect to each other so as to separate fluid with the loose cells and cellular fragments into separate aliquots. The plurality of collection receptacles are also separable from each other after the fluid with the loose cells and cellular fragments has been separated into the separate aliquots.

A second aspect of the present disclosure is to provide a method for recovering cells and cellular components. The method includes placing a biopsy bag with a tissue specimen at least partially within a first sieve collection receptacle, causing fluid that has been mixed with the tissue specimen to flow through a first sieve collection receptacle, through a second sieve collection receptacle, and into a fluid collection receptacle such that the fluid with loose cells and cellular fragments from the tissue sample separates into separate aliquots, and retrieving the separate aliquots from at least two of the first sieve collection receptacle, the second sieve collection receptacle and the fluid collection receptacle for further diagnostics.

A third aspect of the present disclosure is to provide another device for recovering cells and cellular components. The device includes a first sieve collection receptacle, a second sieve collection receptacle, and a fluid collection receptacle. The first sieve collection receptacle includes a first inner space and a first sieve surface. The second sieve collection receptacle includes a second inner space and a second sieve surface having a smaller pore size than the first sieve surface. The fluid collection receptacle includes a third inner space. The first sieve collection receptacle, the second sieve collection receptacle, and the fluid collection receptacle are positioned and arranged such that fluid containing loose cells and cellular fragments can be separated with the first sieve collection receptacle containing cells dislodged from harvested tissue, such as friable cells from a tumor mass that has been biopsied, from the fluid within the first inner space, the lower sieve collection receptacle containing cellular debris or single cells from the fluid that has passed through the first sieve surface and into the second inner space, and the fluid collection receptacle containing fluid and exosomes, nucleic acids, proteins, or other analytes that could be useful as cancer biomarkers that has passed through the first sieve surface and the second sieve surface and into the third inner space.

Other objects, features, aspects and advantages of the apparatuses and methods disclosed herein will become apparent to those skilled in the art from the following detailed description, which, taken in conjunction with the annexed drawings, discloses exemplary embodiments of the disclosed apparatuses and methods.

### BRIEF DESCRIPTION OF THE DRAWINGS

Referring now to the attached drawings which form a part of this original disclosure:

FIGS. 1A to 1E illustrate an example of how a primary bladder cancer tumor is resected and excised biopsy specimens are received;

FIGS. 2A to 2E illustrate an example of how a pathology laboratory prepares specimens for processing by FFPE;

FIG. 3 illustrates examples of three different biopsy bags;

FIG. 4 illustrates a top perspective view of an example embodiment of a device for recovering cells and cellular components in accordance with the present disclosure;

FIG. 5 illustrates a cross-sectional view of the device of FIG. 4;

FIG. 6 illustrates another cross-sectional view of parts of the device of FIG. 4 being pivoted with respect to each other;

FIG. 7 illustrates example embodiment of a method of recovering cells and cellular components in accordance with the present disclosure;

FIG. 8 illustrates an example embodiment of the device of FIGS. 4 to 6 being used during the method of FIG. 7;

FIG. 9 illustrates an example embodiment of the device of FIGS. 4 to 6 being used during the method of FIG. 7;

FIG. 10 illustrates an example embodiment of the device of FIGS. 4 to 6 being used during the method of FIG. 7;

FIG. 11 illustrates an example embodiment of the device of FIGS. 4 to 6 being used during the method of FIG. 7;

FIG. 12 illustrates an example embodiment of the device of FIGS. 4 to 6 being used during the method of FIG. 7;

FIG. 13 illustrates an example embodiment of the device of FIGS. 4 to 6 being used during the method of FIG. 7;

FIG. 14 illustrates an example embodiment of the device of FIGS. 4 to 6 being used during the method of FIG. 7;

FIG. 15 illustrates an example embodiment of the device of FIGS. 4 to 6 being used during the method of FIG. 7;

FIG. 16 illustrates a top perspective view of another example embodiment of a device for recovering cells and cellular components in accordance with the present disclosure being used during the method of FIG. 7;

FIG. 17 illustrates another top perspective view of the device of FIG. 16 being used during the method of FIG. 7;

FIG. 18 illustrates a top perspective view of another example embodiment of a device for recovering cells and cellular components in accordance with the present disclosure being used during the method of FIG. 7;

FIG. 19 illustrates another top perspective view of the device of FIG. 16 being used during the method of FIG. 7;

FIGS. 20A to 20D illustrate a cross-sectional view of another example embodiment of a device for recovering cells and cellular components in accordance with the present disclosure being used during the method of FIG. 7;

FIGS. 21A to 21F illustrate research demonstrating the effectiveness of the device and/or method disclosed herein;

FIG. 22 illustrates a top perspective view of an example embodiment of a device for recovering cells and cellular components in accordance with the present disclosure;

FIG. 23 illustrates a cross-sectional view taken through the center of FIG. 22;

FIG. 24 illustrates a top plan view of an example embodiment of a base receptacle in accordance with the present disclosure;

FIG. 25 illustrates another cross-sectional view taken through the center of FIG. 22;

FIG. 26 illustrates a detailed view of a portion of the device of FIG. 22;

FIG. 27 illustrates a top perspective view of an example embodiment of a biopsy bag supporting device in accordance with the present disclosure;

FIG. 28 illustrates a bottom perspective view of the biopsy bag supporting device of FIG. 27;

FIG. 29 illustrates a top perspective view of the device of FIG. 22;

FIG. 30 illustrates another top perspective view of the device of FIG. 22 with certain parts detached;

FIG. 31 illustrates another top perspective view of the device of FIG. 22 with certain parts detached;

FIG. 32 illustrates another top perspective view of the device of FIG. 22;

FIG. 33 illustrates a top perspective view of an example embodiment of a device for recovering cells and cellular components in accordance with the present disclosure;

FIG. 34 illustrates a cross-sectional view taken through the center of FIG. 33;

FIG. 35 illustrates another top perspective view of the device of FIG. 33;

FIG. 36 illustrates a side perspective view of an example embodiment of a biopsy bag supporting device in accordance with the present disclosure;

FIG. 37 illustrates a top perspective view of an example embodiment of a device for recovering cells and cellular components in accordance with the present disclosure;

FIG. 38 illustrates a cross-sectional view taken through the center of FIG. 37;

FIG. 39 illustrates a detailed view of a portion of a biopsy bag supporting device in accordance with the present disclosure;

FIG. 40 illustrates a detailed view of a portion of a biopsy bag supporting device in accordance with the present disclosure;

FIG. 41 illustrates a bottom perspective view of an example embodiment of a biopsy bag supporting device in accordance with the present disclosure;

FIG. 42 illustrates a top perspective view of an example embodiment of a device for recovering cells and cellular components in accordance with the present disclosure;

FIG. 43 illustrates another top perspective view of the device of FIG. 42 with the parts detached;

FIG. 44 illustrates a detailed view of another example embodiment of a device for recovering cells and cellular components in accordance with the present disclosure;

FIG. 45 illustrates a bottom perspective view of the biopsy bag supporting device shown in FIG. 44;

FIG. 46 illustrates a detailed view of another example embodiment of a device for recovering cells and cellular components in accordance with the present disclosure;

FIG. 47 illustrates a bottom perspective view of the biopsy bag supporting device shown in FIG. 47;

FIG. 48 illustrates a perspective view of another example embodiment of a device for recovering cells and cellular components in accordance with the present disclosure;

FIG. 49 illustrates another perspective view of the device of FIG. 48;

FIG. 50 illustrates a cross-sectional view of the device of FIG. 48;

FIG. 51 illustrates a perspective view of another example embodiment of a device for recovering cells and cellular components in accordance with the present disclosure;

FIG. 52 illustrates another perspective view of the device of FIG. 51;

FIG. 53 illustrates another perspective view of the device of FIG. 51;

FIG. 54 illustrates a cross-sectional view of the device of FIG. 51;

FIG. 55 illustrates a cross-sectional view of another example embodiment of a device for recovering cells and cellular components in accordance with the present disclosure;

FIG. 56A to 56E illustrate the process of using the device of FIG. 55;

FIG. 57 illustrates a perspective view of another example embodiment of a device for recovering cells and cellular components in accordance with the present disclosure;

FIG. 58 illustrates a cross-sectional view of the device of FIG. 57; and

FIG. 59 illustrates example embodiment of a method of recovering cells and cellular components in accordance with the present disclosure.

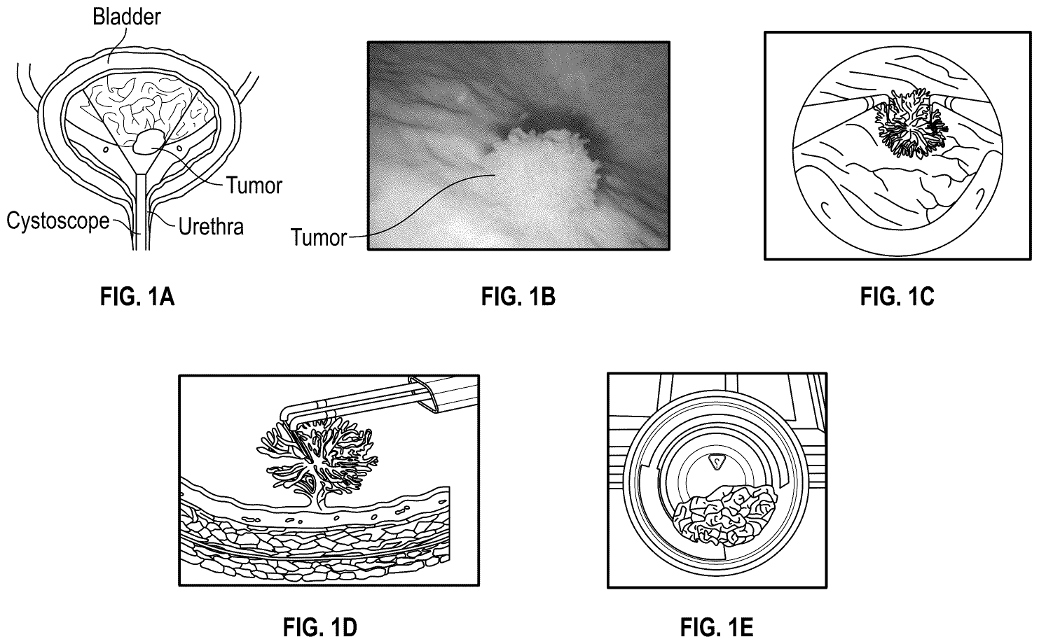

### DETAILED DESCRIPTION OF EMBODIMENTS

Selected embodiments will now be explained with reference to the drawings. It will be apparent to those skilled in the art from this disclosure that the following descriptions of the embodiments are provided for illustration only and not for the purpose of limiting the invention as defined by the appended claims and their equivalents.

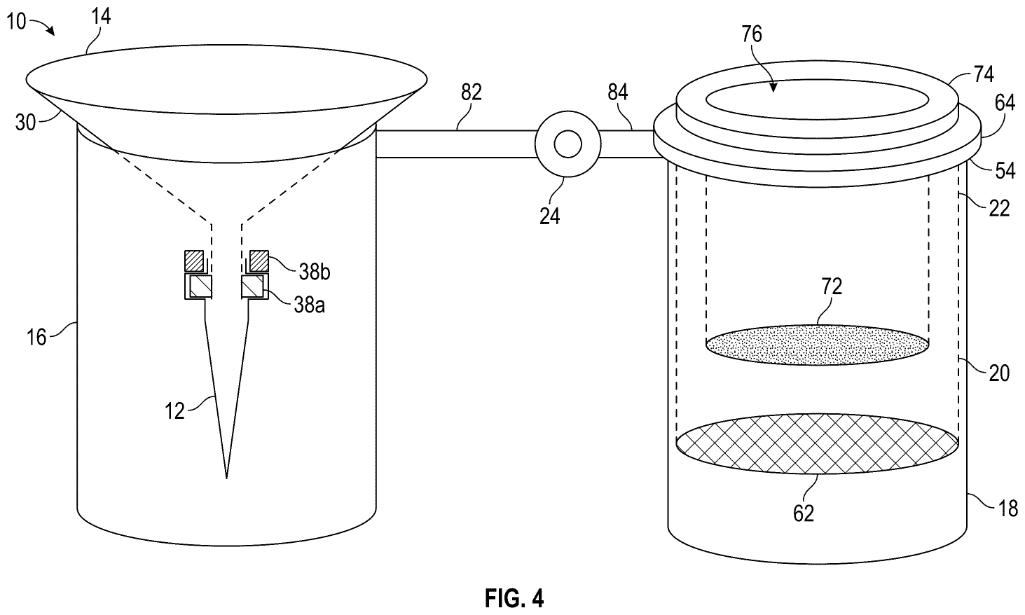

FIGS. 4 to 6 illustrate a first example embodiment of a system or device 10 for recovering cells and cellular components in accordance with the present disclosure. As discussed in more detail below, the device 10 is configured to separate fluid with the loose cells and cellular fragments into three separate aliquots.

The device 10 includes one or more of a biopsy bag 12, a biopsy bag supporting device 14, a base receptacle 16, and a plurality of collection receptacles 18, 20, 22. In the illustrated embodiment, the plurality of collection receptacles 18, 20, 22 includes a fluid collection receptacle 18, a first or lower sieve collection receptacle 20 and a second or upper sieve collection receptacle 22. As discussed in more detail below, the plurality of collection receptacles are positioned and arranged with respect to each other such that pouring fluid with the loose cells and cellular fragments from the base receptacle 16 into one of the plurality of collection receptacles 18, 20, 22 causes separation of the fluid with the loose cells and cellular fragments into three separate aliquots A1, A2, A3 each in a respective collection receptacle 18, 20, 22.

In different embodiments, the biopsy bag 12, the biopsy bag supporting device 14, the base receptacle 16, the fluid collection receptacle 18, the lower sieve collection receptacle 20 and the upper sieve collection receptacle 22 can be separate parts or can be removably attachable to each other. In the illustrated embodiment, the plurality of collection receptacles 18, 20, 22 can be separated from each other after fluid with the loose cells and cellular fragments has been separated into the three separate aliquots A1, A2, A3 as disclosed herein. In the illustrated embodiment, the device 10 also includes an attachment mechanism 24 that moveably attaches the base receptacle 16 to the fluid collection receptacle 18, the biopsy bag 12 and the biopsy bag supporting device 14 are separate parts that are removably insertable into the base receptacle 16, and the lower sieve collection receptacle 20 and the upper sieve collection receptacle 22 are separate parts that are removably insertable into the fluid collection receptacle 18.

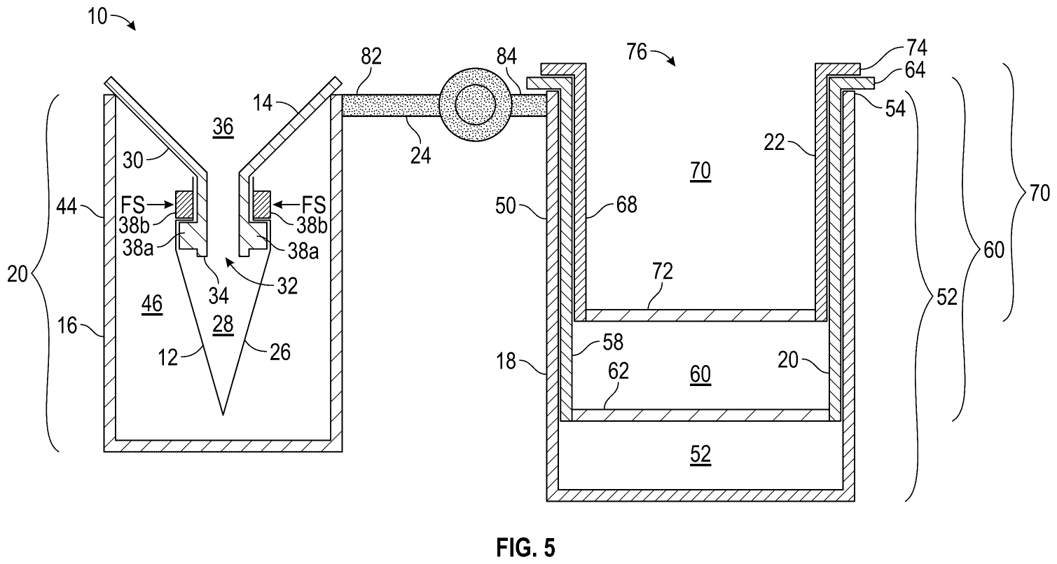

In the illustrated embodiment, the device 10 includes the biopsy bag 12. As seen in FIG. 5, the biopsy bag 12 has an outer surface 26 and an inner space 28. The outer surface 26 includes pores, for example, having a pore size of 150 to 225 micrometers. In various embodiments, the device 10 can include a plurality of different sized biopsy bags 12, or biopsy bags 12 provided outside of the device 10 can be used with the device 10. The device 10 is configured to accommodate various biopsy bags of different sizes.

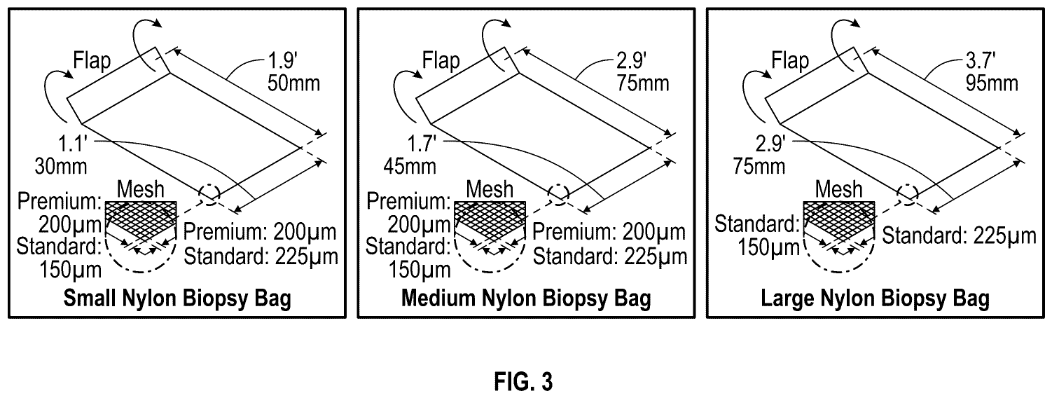

FIG. 3 illustrates three examples of biopsy bags 12 configured to be used in the device 10 in accordance with the present disclosure. The small biopsy bag is approximately 30 mm×50 mm and has a pore size of 150 μm, 200 μm or 225 μm. The medium biopsy bag is approximately 45 mm×75 mm and has a pore size of 150 μm, 200 μm or 225 μm. The large biopsy bag 12c is approximately 75 mm×95 mm and has a pore size of 150 μm or 225 μm. Those of ordinary skill in the art will recognize from this disclosure that the device 10 can accommodate other biopsy bags 12 besides those shown.

In the illustrated embodiment, the device 10 includes the biopsy bag supporting device 14. The biopsy bag supporting device 14 is configured to support the biopsy bag 12 at least partially within the base receptacle 16. As seen in FIGS. 4 and 5, the illustrated biopsy bag supporting device 14 includes a hollow funnel 30 having a conical frustum shape that tapers inwardly towards a dispensing aperture 32 at the lower end 34 thereof. As seen in FIG. 5, the hollow funnel 30 has an interior space 36 that leads to and is in fluid communication with the dispensing aperture 32. The biopsy bag supporting device 14 can also include at least one biopsy bag attachment part 38 operable to removably secure the biopsy bag 12 during the method of use discussed herein.

In the illustrated embodiment, the biopsy bag attachment part 38 includes a first attachment part 38a and a second attachment part 38b. The first attachment part 38a can include one or more protrusion and/or one or more indentation configured to secure the biopsy bag 12 during the method of use discussed herein. In the illustrated embodiment, the first attachment part 38a includes an annular protrusion that encircles the lower end 34 of the hollow funnel 30, and the second attachment part 38b includes an elastic ring that encircles a portion of the lower end 34 to squeeze the biopsy bag 12 onto the biopsy bag supporting device 14, for example, as shown by the squeezing force FS in FIG. 5. As seen in FIG. 5, the second attachment part 38b squeezes the biopsy bag 12 against the lower end 34 at a narrowed diameter location above the first attachment part 38a, thus cooperating with the first attachment part 38a to hold the biopsy bag 12 in place so that the interior space 36 and the dispensing aperture 32 are placed in fluid communication with the inner space 28 of the biopsy bag 12. In other embodiments, the biopsy bag attachment part 38 can include, for example, an annular indentation, one or more spaced protrusions and/or indentations, or other types of clamps or mechanical devices.

In the illustrated embodiment, the device 10 includes the base receptacle 16. The base receptacle 16 includes an outer surface 44 and an inner space 46 within the outer surface 44. The base receptacle 16 is configured to receive at least a portion of the biopsy bag 12 and/or at least a portion of the biopsy bag supporting device 14 within the inner space 46 during the method of use discussed herein. In the illustrated embodiment, the outer wall 46 of the base receptacle 16 has an upper diameter that is smaller than an outer diameter of the biopsy bag supporting device 14, which allows the biopsy bag supporting device 14 to be suspended above the base receptacle 16 while partially inserted into the inner space 46 of the base receptacle 16 as seen in FIG. 4. In other embodiments, the biopsy bag supporting device 14 can be permanently or removeably attached to the base receptacle 16.

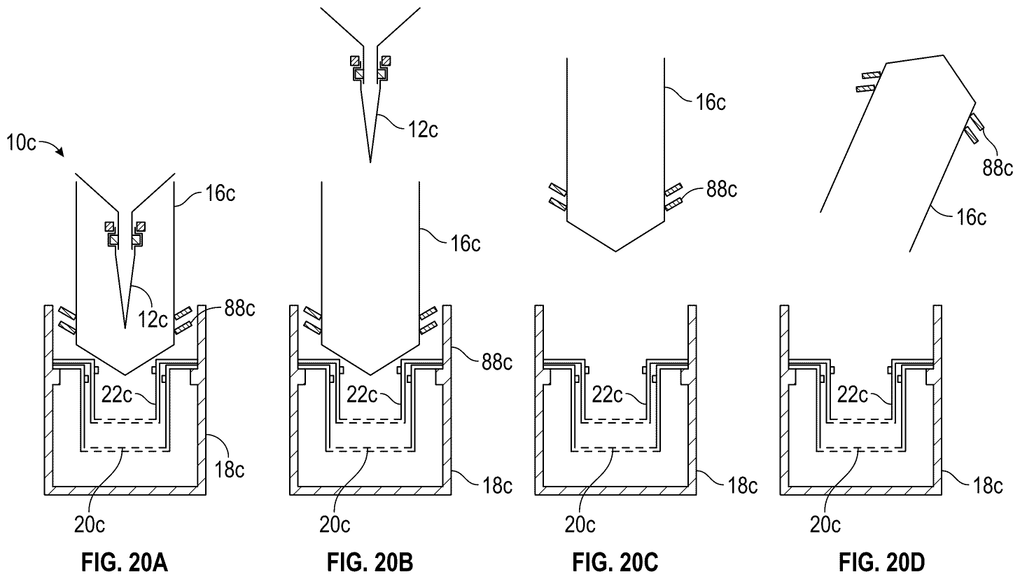

In the illustrated embodiment, the device 10 includes the fluid collection receptacle 18. The fluid collection receptacle 18 includes an outer surface 50 and an inner space 52 within the outer surface 50. The fluid collection receptacle 18 is configured to receive and/or support the lower sieve collection receptacle 20 and the upper sieve collection receptacle 22 at least partially within the inner space 52. As seen in FIG. 5, the fluid collection receptacle 18 can have an edge 54 that supports the lower sieve collection receptacle 20 and the upper sieve collection receptacle 22 such that the lower collection receptacle 20 is suspended at least partially within the fluid collection receptacle 18 and the upper sieve collection receptacle 22 is suspended at least partially within the fluid collection receptacle 18 and the lower sieve collection receptacle 20. In FIGS. 4, 8 to 14 and 16 to 19, broken lines are used to illustrate parts of the lower sieve collection receptacle 20 and the upper sieve collection receptacle 22 that are located within the fluid collection receptacle 18 and/or the lower sieve collection receptacle 20. In the illustrated embodiment, the edge 54 is the upper edge of the outer surface 50, but the edge 54 can also be located in other areas, for example, further down the outer surface 50 within the inner space 50, as shown for example in the alternative embodiment of FIG. 20.

In the illustrated embodiment, the device 10 includes the lower sieve collection receptacle 20. The lower sieve collection receptacle 20 includes an outer surface 58 and an inner space 60 within the outer surface 58. The lower sieve collection receptacle 20 also includes a sieve surface 62. In the illustrated embodiment, the sieve surface 62 is the lower surface or a part of the lower surface of the lower sieve collection receptacle 20. In the illustrated embodiment, the sieve surface has a pore diameter of approximately 5 microns. In other embodiments, the sieve surface 62 can have a pore diameter of approximately 3-8 microns. As discussed in more detail below, the lower sieve collection receptacle 20 is configured to retain cell fragments, red blood cells or other debris during the method of use discussed herein, with fluid including exosomes flowing through the sieve surface 62 and into the fluid collection receptacle 18.

In the illustrated embodiment, the lower sieve collection receptacle 20 also includes a suspension part 64 that enables the lower sieve collection receptacle 20 to be suspended at least partially within the fluid collection receptacle 18. The illustrated suspension part 64 includes an annular ring around the outer circumference of the lower sieve collection receptacle 20. The annular ring has an outer diameter that is larger than that of the edge 54 of the fluid collection receptacle 18, thereby enabling the lower sieve collection receptacle 20 to be suspended at least partially within the inner space 52 the fluid collection receptacle 18 as shown in FIGS. 4 and 5. Those of ordinary skill in the art will recognize from this disclosure that other types of suspension parts 64 can be used to enable the lower sieve collection receptacle 20 to be suspended at least partially within the fluid collection receptacle 18. As illustrated, the lower sieve collection receptacle 20 has a shorter height and a smaller horizontal width/diameter than the fluid collection receptacle 18.

In the illustrated embodiment, the device 10 includes the upper sieve collection receptacle 22. The upper sieve collection receptacle 22 includes an outer surface 68 and an inner space 70 within the outer surface 68. The upper sieve collection receptacle 22 also includes a sieve surface 72. In the illustrated embodiment, the sieve surface 72 is the lower surface or a part of the lower surface of the upper sieve collection receptacle 22. In the illustrated embodiment, the sieve surface 72 has a pore diameter of approximately 20 microns. In other embodiments, the sieve surface 72 can have a pore diameter of approximately 18-30 microns. The sieve surface 72 of the upper sieve collection receptacle 22 thus has a larger pore size than the sieve surface 62 of the lower sieve collection receptacle 20. As discussed in more detail below, the upper sieve collection receptacle 22 is configured to retain friable cells during the method of use discussed herein, with fluid including cell fragments, red blood cells or other debris flowing through the sieve surface 72 and into the lower sieve collection receptacle 20.

In the illustrated embodiment, the upper sieve collection receptacle 22 also includes a suspension part 74 that enables the upper sieve collection receptacle 22 to be suspended at least partially within the lower sieve collection receptacle 20. The illustrated suspension part 74 includes an annular ring around the outer circumference of the upper sieve collection receptacle 22. The annular ring has an outer diameter that is larger than that of an edge (here, the upper edge formed by the suspension part 64) of the lower sieve collection receptacle 20, thereby enabling the upper sieve collection receptacle 22 to be suspended at least partially within the inner space 60 the lower sieve collection receptacle 20 as shown in FIGS. 4 and 5. Those of ordinary skill in the art will recognize from this disclosure that other types of suspension parts 74 can be used to enable the upper sieve collection receptacle 22 to be suspended at least partially within the lower sieve collection receptacle 20 and/or the fluid collection receptacle 18. As illustrated, the upper sieve collection receptacle 22 has a shorter height and a smaller horizontal width/diameter than the lower sieve collection receptacle 20.

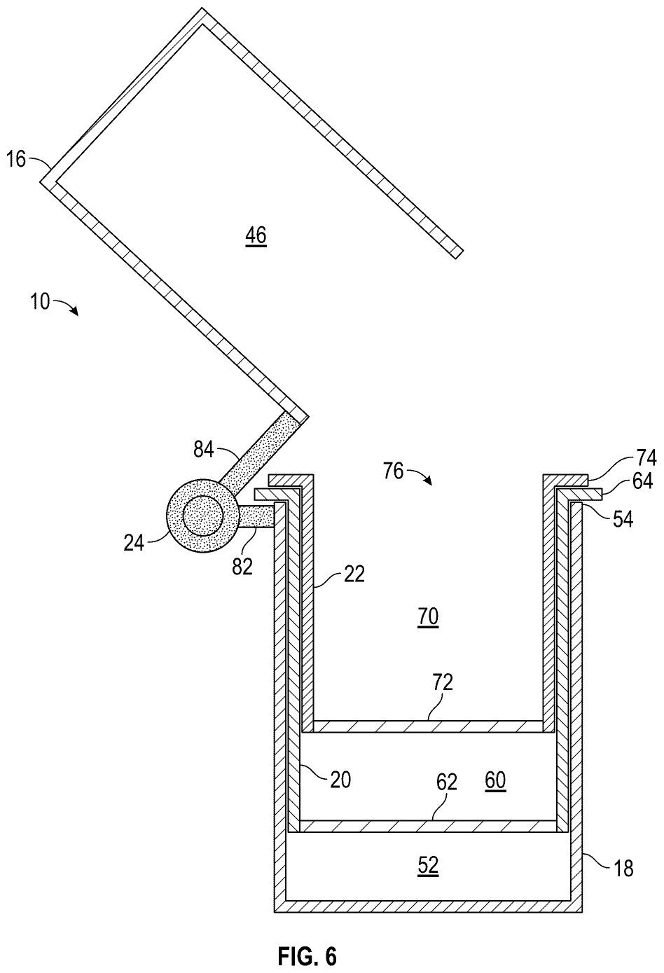

In the illustrated embodiment, the base receptacle 16 is moveably attached to the fluid collection receptacle 18. More specifically, the base receptacle 16 is pivotably attached to the fluid collection receptacle 18 by an attachment mechanism 24. In the embodiment shown in FIGS. 4 to 6, the attachment mechanism 24 that connects the base receptacle 16 to the fluid collection receptacle 18 includes a first part 82 and a second part 84 that pivot with respect to each other. The first part 82 is attached to the base receptacle 16 at its outer surface 44, and the second part 84 is attached to the fluid collection receptacle 18 at its outer surface 50, to enable the base receptacle 16 to pivot with respect to the fluid collection receptacle 18 while staying attached to the fluid collection receptacle 18, as seen for example in FIG. 6. As discussed in more detail below, this movement enables fluid with loose cells and cellular fragments from a tissue specimen to be poured from the base receptacle 16 into the fluid collection receptacle 18.

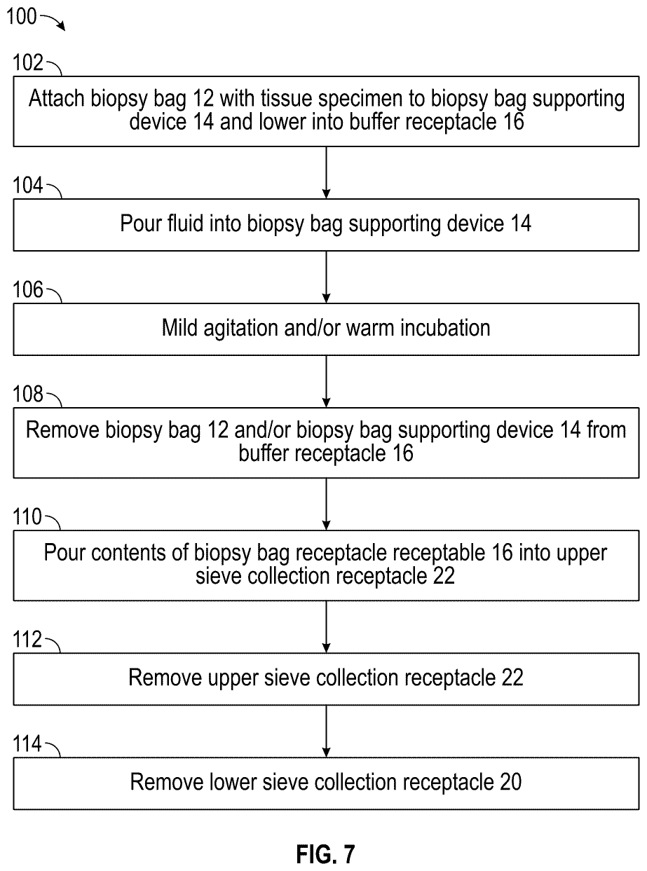

FIG. 7 illustrates an example embodiment of a method 100 of recovering cells and cellular components in accordance with the present disclosure. In an embodiment, the method 100 is a method of using the device 10 described herein to recover cells and cellular components. Those or ordinary skill in the art will recognize from this disclosure, however, that the method 100 can also be practiced with alternative systems and does not necessary require the device 10 shown. Those of ordinary skill in the art will also recognize from this disclosure that certain steps of the method 100 can be added, removed or altered without departing from the spirit and scope of the present disclosure.

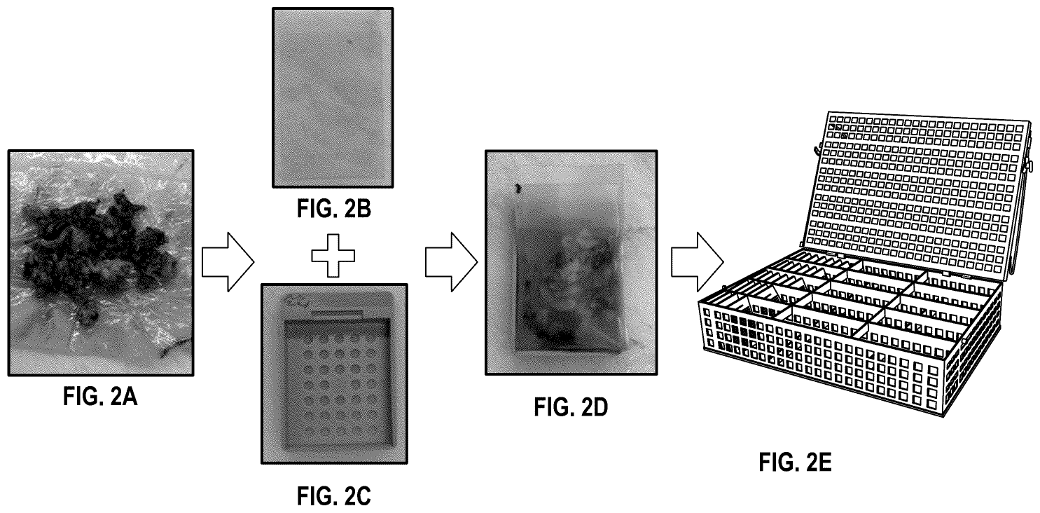

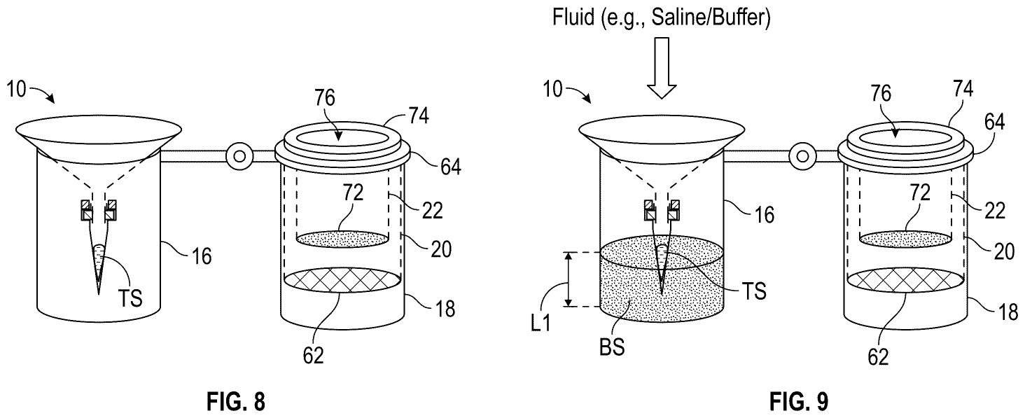

At step 102, when a technician in a clinical pathology lab receives a tissue specimen TS (e.g., a specimen of resected bladder tumor), he or she places it in a biopsy bag 12 (e.g., as seen in FIGS. 2, 2(D)) as usual, but instead of placing the full biopsy bag 12 in a cassette (e.g., FIGS. 2, 2(C), 2(D)) and into a processor box (e.g., FIGS. 2, 2(E)), the biopsy bag 12 is attached to the distal end 34 of the biopsy bag supporting device 14 using the biopsy bag attachment part 38. The assembly of the biopsy bag supporting device 14, the biopsy bag 12 containing the tissue, and the biopsy bag attachment part 38 is lowered into the base receptacle 16, as shown for example in FIG. 8.

At step 104, the technician or other individual pours fluid into the biopsy bag supporting device 14. Here, the fluid includes saline or buffer solution, such as phosphate buffered saline (PBS) solution, but the method 100 could work with other types of solution. In the illustrated embodiment, the fluid is poured into the hollow funnel 30, so that it flows through the dispensing aperture 32 and into the biopsy bag 12, and then through the biopsy bag pores and into the base receptacle 16. The fluid should be poured so that it rises to a level L1 that covers the tissue specimen TS in the biopsy bag 12, as shown for example in FIG. 9. In an embodiment, step 104 can be performed without a biopsy bag supporting device 14, for example, by suspending the biopsy bag 12 at least partially within the base receptacle 16 in another manner while pouring the buffered saline BS into the biopsy bag 12.

At step 106, the technician or other individual applies mild agitation and/or warm incubation to encourage loose cells to release exosomes and to disperse through the holes of the biopsy bag 12 into the fluid in the base receptacle 16. In an embodiment, the mild agitation and/or warm incubation can be performed for approximately five minutes. This creates a fluid F which combines the buffered saline BS with loose cells and cellular fragments in the base receptacle 16. It should also be understood from this disclosure that some loose cells and cellular fragments will flow with the fluid into the base receptacle 16 even before any mild agitation and/or warm incubation.

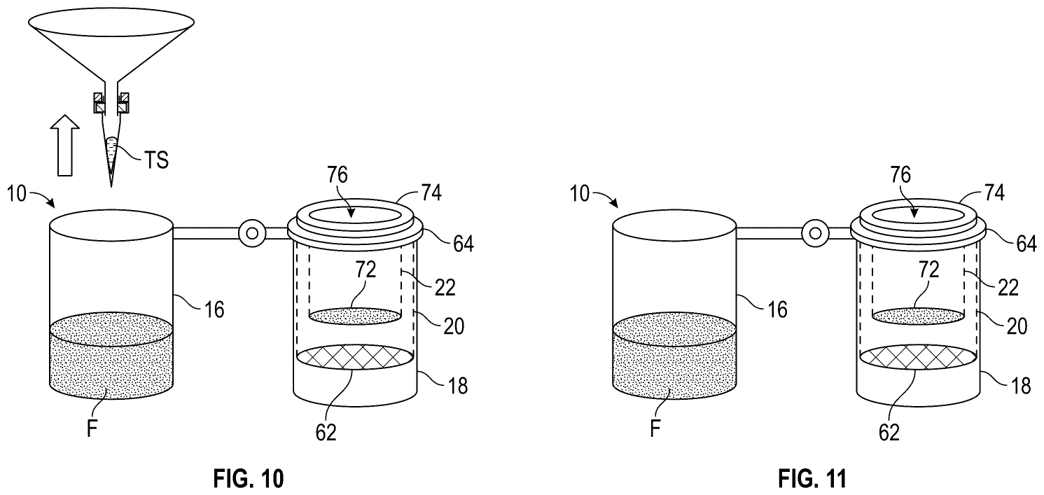

At step 108, the assembly of the biopsy bag supporting device 14, the biopsy bag 12 containing the tissue, and the biopsy bag attachment part 38 can be lifted out of the base receptacle 16, as shown for example in FIG. 10. The tissue specimen TS can be kept in the biopsy bag 12 and moved to a standard cassette for FFPE processing and microscopic examination which is the standard process today.

At step 110, after the biopsy bag 12 has been removed, the base receptacle 16 still contains the fluid F including solution with loose cells and cellular fragments from the tissue specimen, as shown for example in FIG. 11. The technician or other individual then pours the fluid F from the base receptacle 16 into the upper opening 76 of the upper sieve collection receptacle 22, thereby causing the fluid F containing the loose cells and cellular fragments to flow sequentially into the inner space 70 of the upper sieve collection receptacle 22, then through the sieve surface 72 of the upper sieve collection receptacle 22 and into the inner space 60 of the lower sieve collection receptacle 20, then through the sieve surface 62 of the lower sieve collection receptacle 20 and into the inner space 52 of the fluid collection receptacle 18. The fluid F including buffered saline solution with loose cells and cellular fragments from the tissue is thus poured into each of the fluid collection receptacle 18, the lower sieve collection receptacle 20 and the upper sieve collection receptacle 22 by being poured into the upper sieve collection receptacle 24.

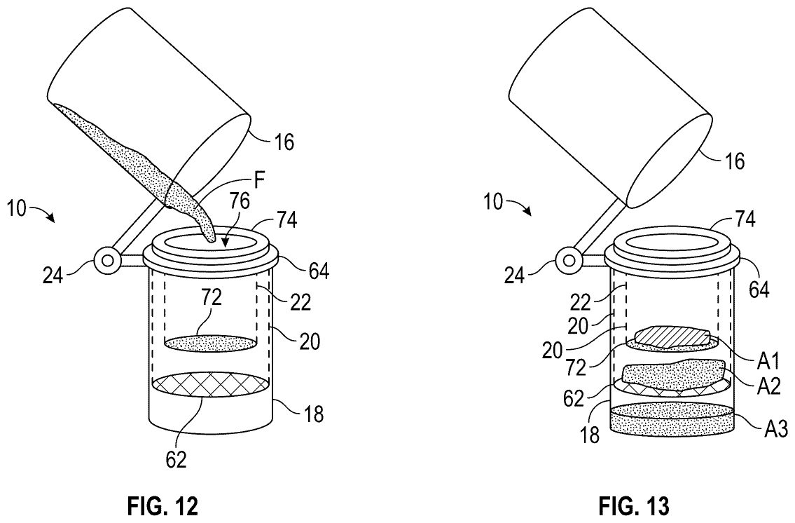

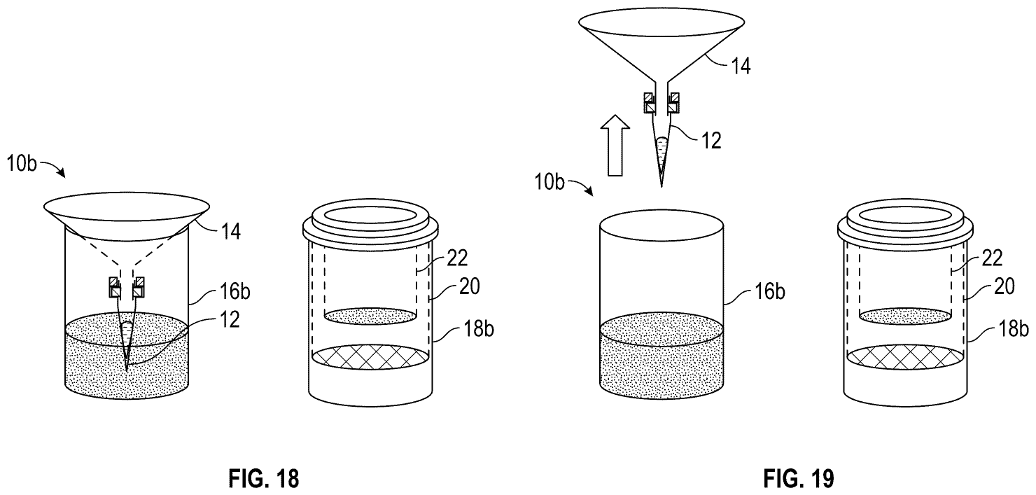

FIG. 12 illustrates an example embodiment of how the technician or other individual pours the fluid F from the base receptacle 16 into the upper opening 76 of the upper sieve collection receptacle 22 by pivoting the base receptacle 16 with respect to the fluid collection receptacle 18 using the attachment mechanism 24. Those of ordinary skill in the art will also recognize from this disclosure that this step can also be performed with a base receptacle 16 and a fluid collection receptacle 18 that are separate from each other or attached by another mechanism, as shown for example at FIGS. 18 and 19.

After step 110, the fluid collection receptacle 18, the lower sieve collection receptacle 20 and the upper sieve collection receptacle 22 should contain different aliquots A1, A2, A3. This is the result of pouring the fluid F from the base receptacle 16 and into the upper sieve collection receptacle 22, and through the sieve surface 72 of the upper sieve collection receptacle 22 and the sieve surface 62 of the lower sieve collection receptacle 20, so that the fluid F separates into three aliquots A1, A2, A3, as shown for example in FIG. 13. The technician or other individual should allow sufficient time for the fluid F to separate into the three aliquots A1, A2, A3. In an embodiment, the fluid F can be pulled downward with the use of a common suction attachment such as a Buchner funnel apparatus.

As seen in FIG. 13, the upper sieve collection receptacle 22 collects friable cells (the first aliquot A1) that have fallen off from the solid (“parent”) tissue specimen TS. These include in-tact cells that can be used, for example, for metabolomic studies (via extraction of metabolites with 80% methanol), genomics studies. organoid growth, and/or nucleic acid (DNA and RNA) analysis. RNA analysis is sometimes also called transcriptomics. In the embodiment shown, the pore size of the sieve surface 72 is approximately 20 microns, so the upper sieve collection receptacle 22 captures cells that are larger than 20 microns. In other embodiments, the sieve surface 72 can have a pore diameter of approximately 18-30 microns. Those of ordinary skill in the art will recognize from this disclosure that the pore size can vary.

As also seen in FIG. 13, the lower sieve collection receptacle 20 collects cellular debris (the second aliquot A2), and separates it from the final, valuable fluid (the third aliquot A3) that passes through the sieve surface 62 and is collected at the bottom of the fluid collection receptacle 18. The second aliquot A2 includes cell fragments, red blood cells, and debris. This cellular debris currently has no clinical value and can be discarded. Those of ordinary skill in the art should recognize, however, that new uses may arise for the second aliquot A2 in the future. In the embodiment shown, the pore size of the sieve surface 62 is approximately 7 microns, so the lower sieve collection receptacle 20 should capture cellular debris that is larger than 7 microns. In other embodiments, the sieve surface 62 can have a pore diameter of approximately 3-10 microns. Those of ordinary skill in the art will recognize from this disclosure that the pore size can vary as needed or desired.

As also seen in FIG. 13, the fluid collection receptacle 18 collects the final, valuable fluid (the third aliquot A3) that passes through the sieve surface 72 of the upper sieve collection receptacle 22 and the sieve surface 62 of the lower sieve collection receptacle 20. That fluid contains exosomes or the contents of exosomes such as nucleic acids and proteins.

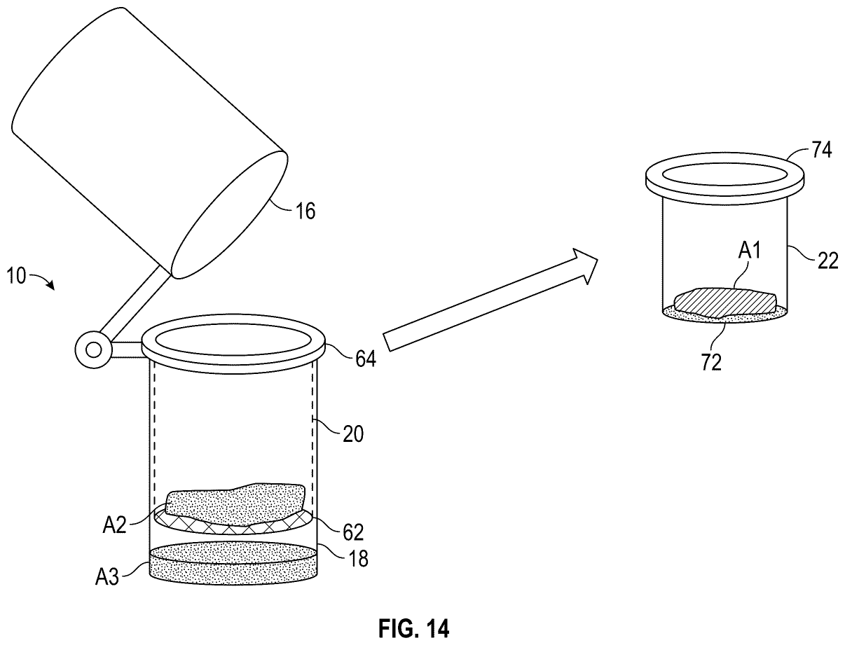

At step 112 of the method 100, the technician or other individual removes the upper sieve collection receptacle 22 from the fluid collection receptacle 18 and the lower sieve collection receptacle 20, as seen for example in FIG. 14. This allows the user to use the recovered in-tact cells, for example, for metabolomics (via extraction of metabolites with 80% methanol), organoid growth, and nucleic acid (DNA and RNA) analysis. In the illustrated embodiment, the upper sieve collection receptacle 22 is removed from the fluid collection receptacle 18 and the lower sieve collection receptacle 20 by simply lifting it out of the lower sieve collection receptacle 20.

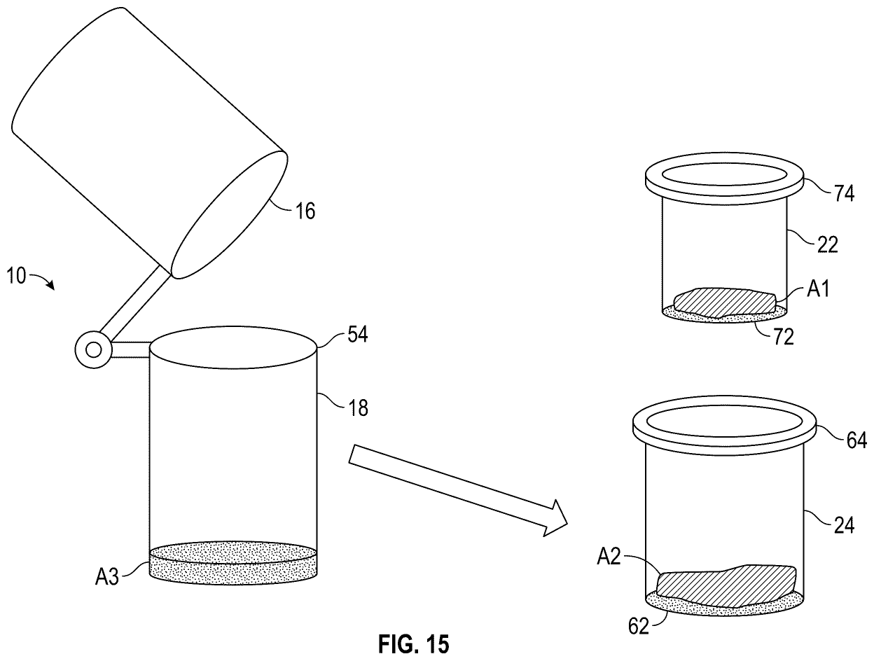

At step 114, the technician or other individual removes the lower sieve collection receptacle 20 from the fluid collection receptacle 18, as seen for example in FIG. 15. In an embodiment, the lower sieve collection receptacle 20 contents (cell fragments, red blood cells and debris) can be discarded. The general purpose of the lower sieve collection receptacle 20 is to remove the cell fragments, red blood cells and debris from the fluid that flows into the fluid collection receptacle 18. The fluid retained in the fluid collection receptacle 18 contains exosomes and can further be used for investigational research studies to correlate exosome associated with certain tumors. The contents of this fluid is far more specific to the tumor than exosome-containing fluids from the body, such as urine, and is therefore an excellent substrate for tumor biomarker analysis.

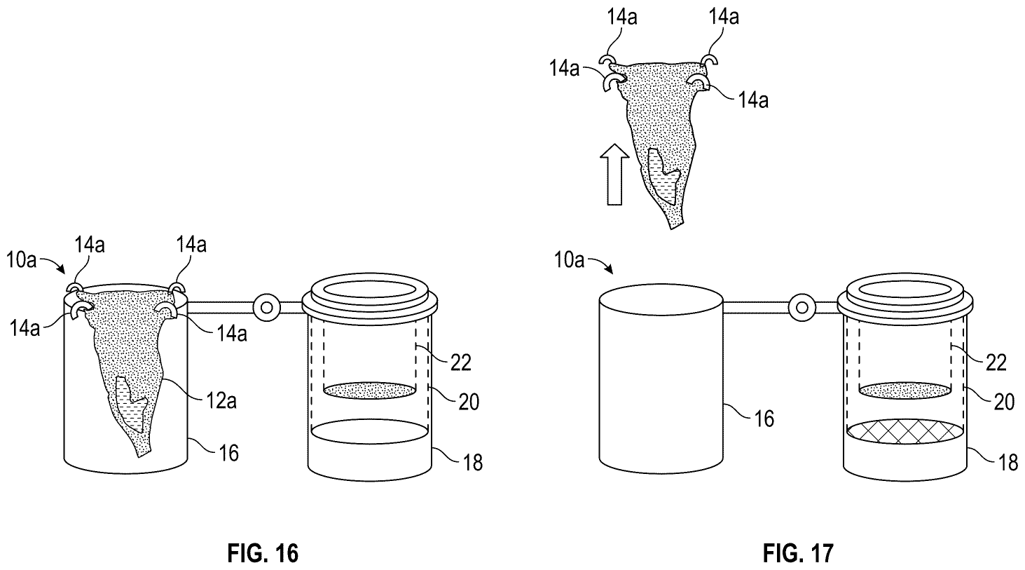

FIGS. 16 and 17 illustrate an alternative embodiment of a device 10a for recovering cells and cellular components in accordance with the present disclosure. As seen in FIGS. 16 and 17, the device 10a utilizes an alternative type of biopsy bag supporting device 14a which suspends the illustrated biopsy bag 12a within the base receptacle 16a without a hollow funnel. Here, the biopsy bag supporting device 14a includes a plurality of clips that suspend the biopsy bag within the base receptacle 16a. The biopsy bag 12a can be removed from the base receptacle 16a, for example, by removing the biopsy bag 12a from the plurality of clips or by releasing the plurality of clips. Those of ordinary skill in the art will recognize from this disclosure that any of the other features of the device 10 can be added to the device 10a illustrated in FIGS. 16 and 17.

FIGS. 18 and 19 illustrate another alternative embodiment of a device 10b for recovering cells and cellular components in accordance with the present disclosure. As illustrated, the device 10b does not include any type of attachment mechanism 24 that moveably attaches the base receptacle 16b to the fluid collection receptacle 18b. Those of ordinary skill in the art will recognize from this disclosure that any of the other features of the device 10 can be added to the device 10b illustrated in FIGS. 18 and 19.

FIG. 20 illustrates another alternative embodiment of a device 10c for recovering cells and cellular components in accordance with the present disclosure. As illustrated, the base receptacle 16c is not connected to the fluid collection receptacle 18c. The base receptacle 16c is instead outfitted with screw threads 88c and sized so that it rests securely but removably inside the upper end of the larger fluid collection receptacle 18a (e.g., as shown at 20(A)). In this embodiment, once the biopsy bag 12c containing the tissue specimen is removed from the fluid in the base receptacle 16c (e.g., as shown at 20(B)), the user unscrews the base receptacle 16c from its position (e.g., as shown at 20(C)) and inverts it (e.g., as shown at 20(D)), pouring the fluid into the larger fluid collection receptacle 18c. The fluid flows from the base receptacle 16c into the upper sieve collection receptacle 22c, the lower sieve collection receptacle 20c, and the fluid collection receptacle 18c, and as discussed herein with respect to the device 10.

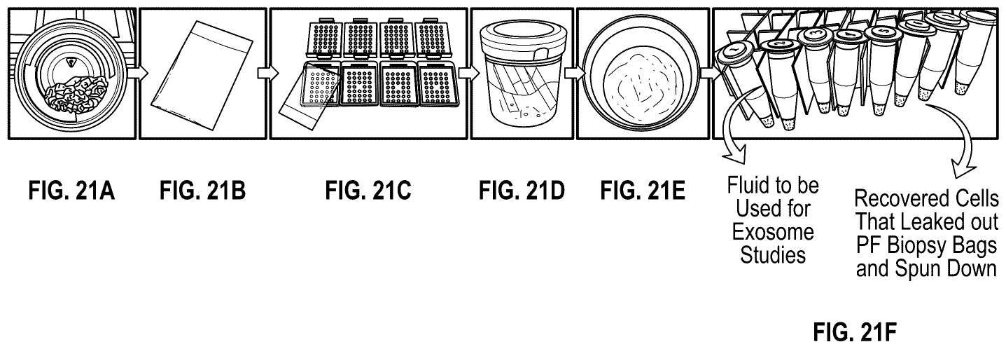

FIG. 21 illustrates preliminary research demonstrating the effectiveness of the device 10 and/or method 100 disclosed herein. Image 21(A) shows a TURBT specimen with bladder cancer tissue received from an operation room. The tissue was placed in biopsy bags (21(D)), which was then placed in cassettes (21(C)) and then into another specimen cup with PBS buffer (21(D)). The contents were gently mixed for five minutes and the cassettes removed and transferred for FFPE processing leaving a cloudy fluid in the specimen cup (21(E)). The fluid was then spun down, resulting in a surprisingly large amount of recoverable cells (21(F)) (seen a pellets at the bottom of the microcentrifuge tubes). It is thus believed that such a collection method can be useful for creating aliquots for multiple different analytes for different technologies/assays, for example, examining the supernatant for presence of exosomes, methanol extraction for metabolic studies, growing organoids and/or for the extraction of nucleic acids.

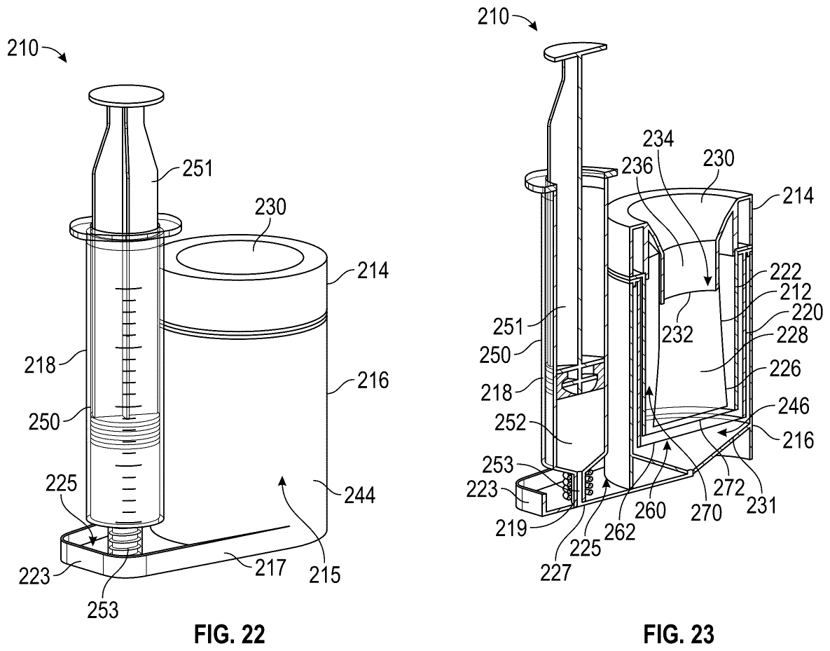

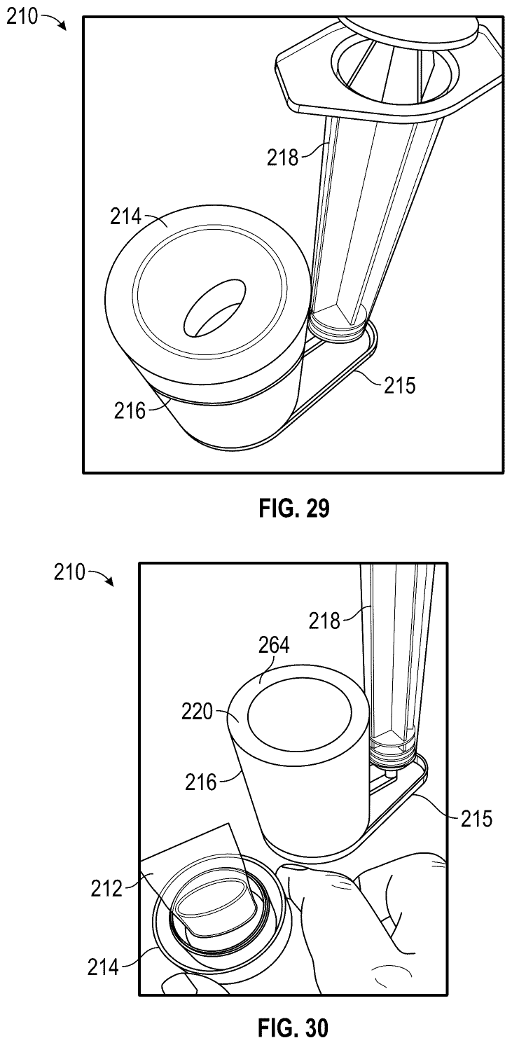

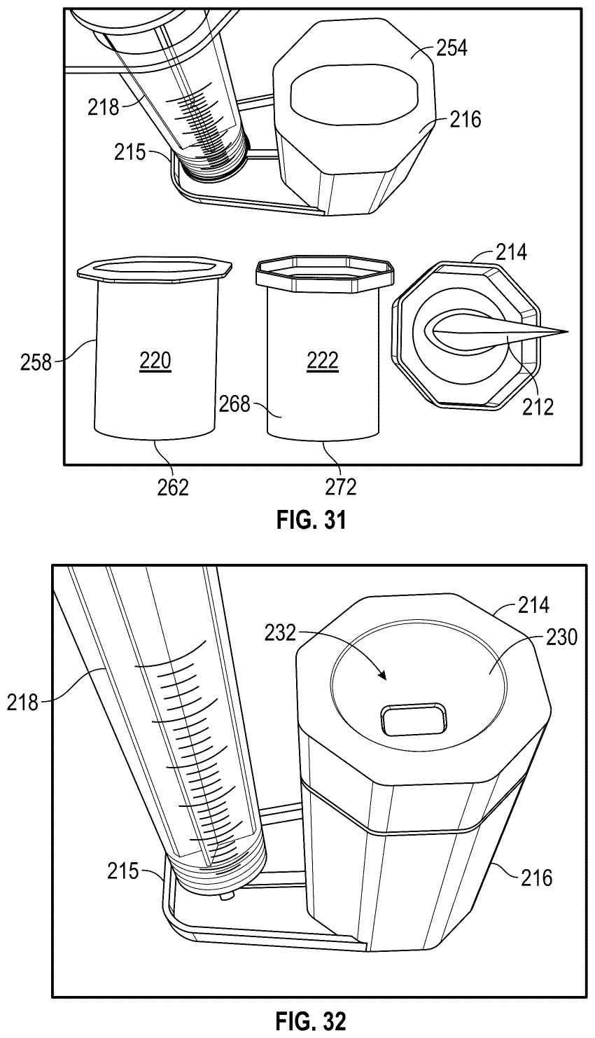

FIGS. 22 to 32 illustrate another example embodiment of system or device 210 for recovering cells and cellular components in accordance with the present disclosure. As with the previous embodiments, the device 210 is configured to separate fluid with the loose cells and cellular fragments into separate aliquots. Specifically, the device 210 is configured to recover eight analyte types that are central to cancer detection research: exosomes, metabolites, proteins, RNA, high-quality DNA and viable cells suitable for organoid growth, from bladder cancers resected as TURBTs. These valuable molecular and cellular material can be used in eight fields of study: (1) genomics, (2) epigenomics, (3) transcriptomics, (4) metabolomics, (5) single-cell-omics, (6) proteomics, (7) exome research, and (8) organoid growth.

As with the previous embodiments, the device 210 includes one or more of a biopsy bag 212, a biopsy bag supporting device 214, a base receptacle 216, and a plurality of collection receptacles 218, 220, 222. In the illustrated embodiment, the plurality of collection receptacles 218, 220, 222 includes a fluid collection receptacle 218, a first or lower sieve collection receptacle 220 and a second or upper sieve collection receptacle 222. As with the previous embodiments, the plurality of collection receptacles are positioned and arranged to cause separation of fluid with the loose cells and cellular fragments into three separate aliquots A1, A2, A3 each in a collection receptacle 218, 220, 222.

As with the previous embodiments, the biopsy bag 212, the biopsy bag supporting device 214, the base receptacle 216, the fluid collection receptacle 218, the lower sieve collection receptacle 220 and the upper sieve collection receptacle 222 can be separate parts that are removably attachable to each other. More specifically, the plurality of collection receptacles 218, 220, 222 can be separated from each other after fluid with the loose cells and cellular fragments has been separated into the three separate aliquots A1, A2, A3 as disclosed herein.

As with the previous embodiments, the biopsy bag 212 has an outer surface 226 and an inner space 228. The outer surface 226 includes pores, for example, having a pore size of 150 to 225 micrometers. As with the previous embodiments, those of ordinary skill in the art will recognize from this disclosure that the device 210 can accommodate the biopsy bags 12 shown in FIG. 3 or other biopsy bags 12, 212 besides those shown.

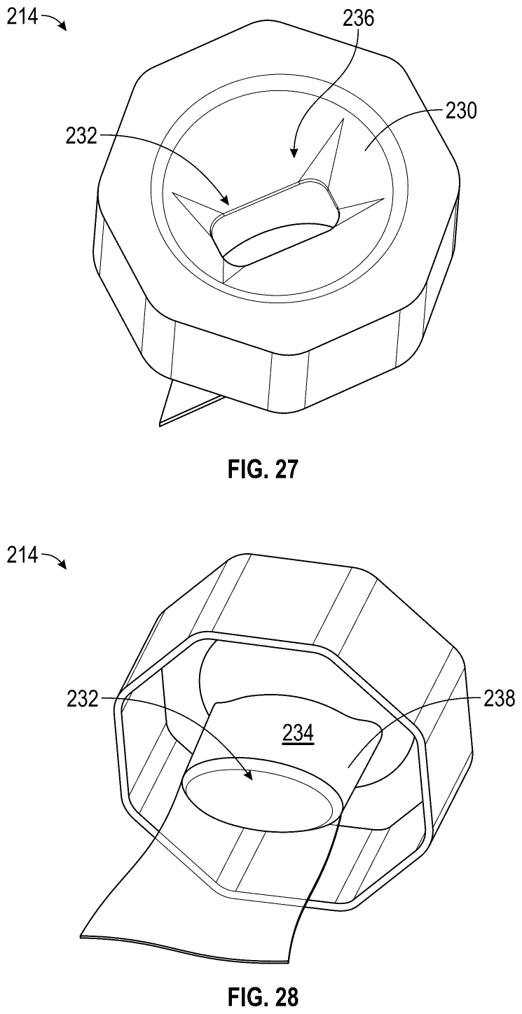

As with the previous embodiments, the device 210 includes a biopsy bag supporting device 214. The biopsy bag supporting device 214 is configured to support the biopsy bag 212 at least partially within the base receptacle 216, the lower sieve collection receptacle 220, and/or the upper sieve collection receptacle 222, as shown for example in FIG. 23. As seen in more detail in FIGS. 27 and 28, the biopsy bag supporting device 214 includes a hollow funnel 230 having a conical frustum shape that tapers inwardly towards a dispensing aperture 232 at the lower end 234 thereof. The hollow funnel 230 has an interior space 236 that leads to and is in fluid communication with the dispensing aperture 232. The biopsy bag supporting device 214 can also include at least one biopsy bag attachment part 238 operable to removably secure the biopsy bag 212 during the method of use discussed herein. As seen in FIGS. 26 and 28, the biopsy bag attachment part 238 includes a downward protrusion surrounding the dispensing aperture 232, with the biopsy bag capable of being slid over the downward protrusion prior to the biopsy bag supporting device 214 being attached to the rest of the device 210 as seen in FIG. 22.

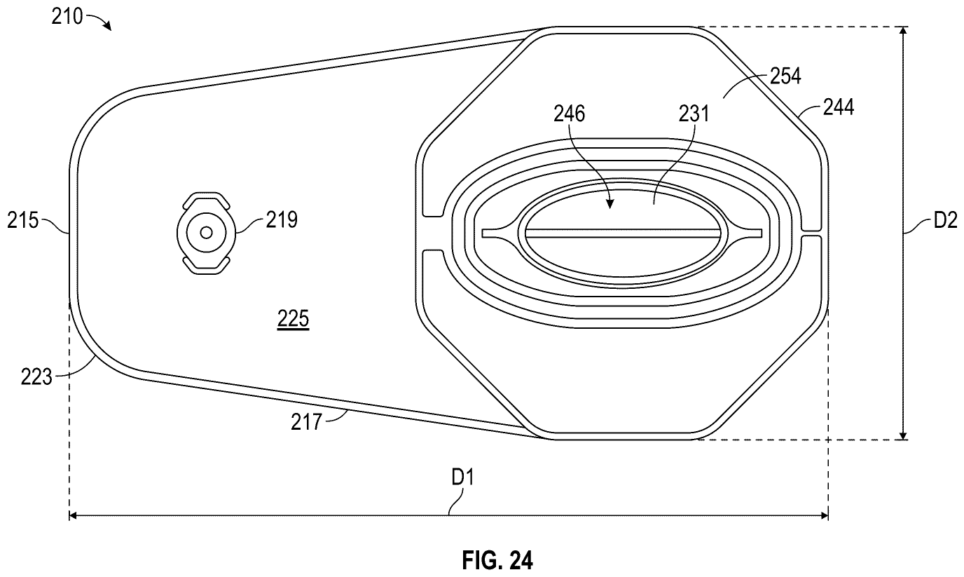

As seen in FIGS. 22 to 24, the device 210 includes a base 215. The base 215 is placed on a flat surface such as a countertop during use of the device 210. In the illustrated embodiment, the base 215 includes the base receptacle 216, a lower support 217 and an attachment port 219. As seen in FIG. 24, the base receptacle 216 is located on one side of the lower support 217, and the attachment port 219 is located on the opposite side of the lower support 217 in the length direction. The lower support 217 further includes an outer wall 223 on the side of the attachment port 219. The outer wall 223 creates a cavity 225 surrounding the attachment port 219 to catch fluid overflow at the attachment port 219. As further seen in FIG. 24, the lower support has a length of distance D1 (e.g., 82.50 mm) that is longer than its width of distance D2 (e.g., 45 mm).

The base receptacle 216 includes an outer wall 244 and an inner space 246 within the outer surface 244. As seen in FIG. 23, the base receptacle 216 is configured to receive at least a portion of the biopsy bag 212 and/or at least a portion of the biopsy bag supporting device 214 within the inner space 246 when constructed as shown herein. The base receptacle 216 is also configured to receive at least a portion of the lower sieve collection receptacle 220 and at least a portion of the upper sieve collection receptacle 222 when constructed as shown herein. As seen in FIG. 26, the outer wall 244 of the base receptacle 16 has an inner edge 254 with a diameter that is smaller than an outer diameter of the biopsy bag supporting device 214, which allows the biopsy bag supporting device 14 to be suspended above the base receptacle 216 while partially inserted into the inner space 246 of the base receptacle 216 as seen in FIG. 23. Similarly, the inner edge 254 has a diameter that is smaller than an outer diameter of each of the lower sieve collection receptacle 220 and the upper sieve collection receptacle 222, which allows the lower sieve collection receptacle 220 and the upper sieve collection receptacle 222 to be suspended above the base receptacle 216 while partially inserted into the inner space 246 of the base receptacle 216 as seen in FIG. 23. The biopsy bag supporting device 214, the lower sieve collection receptacle 220 and the upper sieve collection receptacle 222 are removeably attached to the base receptacle 216. FIG. 26 illustrates in more detail how the biopsy bag supporting device 214, the base receptacle 216, the lower sieve collection receptacle 220 and the upper sieve collection receptacle 222 are attached so as to rest on each other during use.

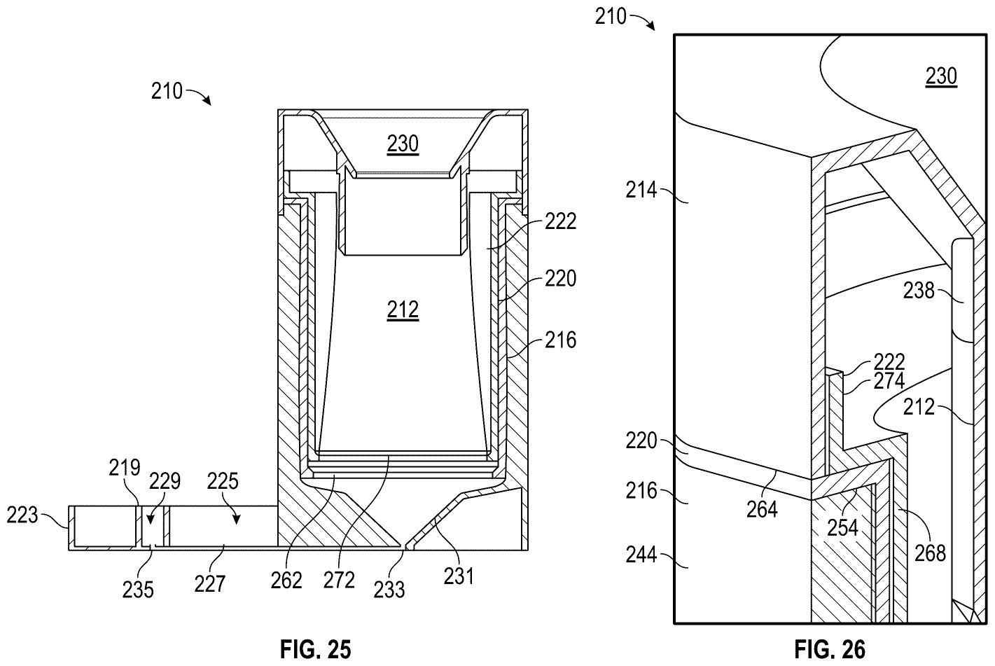

As seen in FIG. 25, the base 215 further includes a fluid channel 227. In the illustrated embodiment, the fluid channel 227 is disposed within the lower support 217. The fluid channel 227 places the inner space 246 of the base receptacle 216 in fluid communication with the inner space 229 of the attachment port 219. In the illustrated embodiment, the base receptacle 216 further includes a funnel portion 231 that funnels into a first end 233 of the fluid channel 227. The opposite second end 235 of the fluid channel 227 is connected to and located below the attachment port 219. Fluid within the inner space 246 of the base receptacle 216 thus flows down the funnel portion 231, so that it can be sucked through the fluid channel 227 and into the fluid collection receptacle 218 via the attachment port 219. The fluid sucked into the fluid collection receptacle 218 can include loose cells, debris, proteins, and nucleic acids that have high diagnostic value in cancer screening research.

In the illustrated embodiment, the fluid collection receptacle 218 is a syringe including an outer surface 250, a piston or plunger 251, an inner space 252 within the outer surface 250, and a tip 253. The fluid collection receptacle 218 is configured to attach to the base 215 at the attachment port 219. For example, the fluid collection receptacle 218 can be attached to the attachment portion 219 by inserting the tip 253 inside the inner space 229 of the attachment port 219. When attached, the tip 253 of the syringe fluidly connects to the fluid channel 227 via the attachment port 219, which places the inner space 246 of the base receptacle 216 in fluid communication with the inner space 252 of the fluid collection receptacle 218.

As with the previous embodiments, the lower sieve collection receptacle 220 includes an outer surface 258 and an inner space 260 within the outer surface 258. The lower sieve collection receptacle 220 also includes a sieve surface 262. In the illustrated embodiment, the sieve surface 262 is the lower surface or a part of the lower surface of the lower sieve collection receptacle 220. In the illustrated embodiment, the sieve surface has a pore diameter of approximately 7 microns. In other embodiments, the sieve surface 262 can have a pore diameter of approximately 3-10 microns. Those of ordinary skill in the art will recognize from this disclosure that the pore size can vary as needed or desired. The lower sieve collection receptacle 220 is configured catch debris but allow proteins and nucleic acids to flow through. The lower sieve collection receptacle 220 is configured to retain cell fragments, red blood cells or other debris during the method of use discussed herein, with fluid including exosomes, nucleic acids, proteins, and other molecular biomarkers flowing through the sieve surface 262, through channel 229, and into the fluid collection receptacle 252, when the user pulls on plunger 210 of syringe 218.

As seen in FIG. 26, the lower sieve collection receptacle 220 also includes a radially extending lip 264 (e.g., suspension part) that enables the lower sieve collection receptacle 220 to be suspended at least partially within the base receptable 216. The lip 264 includes an annular ring around the outer circumference of the lower sieve collection receptacle 220. The annular ring has an outer diameter that is larger than that of the inner edge 254 of the base receptable 216, thereby enabling the lower sieve collection receptacle 220 to rest on and be suspended at least partially within the base receptable 216, as seen in FIGS. 23 and 25. Those of ordinary skill in the art will recognize from this disclosure that other types of suspension parts 264 can be used to enable the lower sieve collection receptacle 220 to be suspended at least partially within the base receptacle 216. As illustrated, the lower sieve collection receptacle 220 has a shorter height and a smaller horizontal width/diameter than the base receptacle 216.

As with the previous embodiments, the upper sieve collection receptacle 222 also includes an outer surface 268 and an inner space 270 within the outer surface 268. The upper sieve collection receptacle 222 also includes a sieve surface 272. In the illustrated embodiment, the sieve surface 272 is the lower surface or a part of the lower surface of the upper sieve collection receptacle 222. In the illustrated embodiment, the sieve surface 272 has a pore diameter of approximately 20 microns. In other embodiments, the sieve surface 272 can have a pore diameter of approximately 18-30 microns. Those of ordinary skill in the art will recognize from this disclosure that the pore size can vary as needed or desired. The sieve surface 272 of the upper sieve collection receptacle 222 thus has a larger pore size than the sieve surface 262 of the lower sieve collection receptacle 220. The upper sieve collection receptacle 222 is configured to catch tumor cells but allows debris, red blood cells, and analytes to flow through. The upper sieve collection receptacle 222 is configured to retain friable cells during the method of use discussed herein, with fluid including cell fragments, red blood cells or other debris flowing through the sieve surface 272 and into the lower sieve collection receptacle 220 when the user pulls on plunger 210 of syringe 218.

As seen in FIG. 26, the upper sieve collection receptacle 222 includes a radially extending lip 274 (e.g., suspension part) that enables the upper sieve collection receptacle 222 to be suspended at least partially within the lower sieve collection receptacle 220. The illustrated suspension part 274 includes an annular ring around the outer circumference of the upper sieve collection receptacle 222. The annular ring has an outer diameter that is larger than that of an edge (here, the upper edge formed by the suspension part 264) of the lip 264 of the lower sieve collection receptacle 220, thereby enabling the upper sieve collection receptacle 222 to rest on and be suspended at least partially within the inner space 260 the lower sieve collection receptacle 220, as seen in FIGS. 23 and 25. Those of ordinary skill in the art will recognize from this disclosure that other types of suspension parts 274 can be used to enable the upper sieve collection receptacle 222 to be suspended at least partially within the lower sieve collection receptacle 220 and/or the fluid collection receptacle 18. As illustrated, the upper sieve collection receptacle 222 has a shorter height and a smaller horizontal width/diameter than the lower sieve collection receptacle 220.

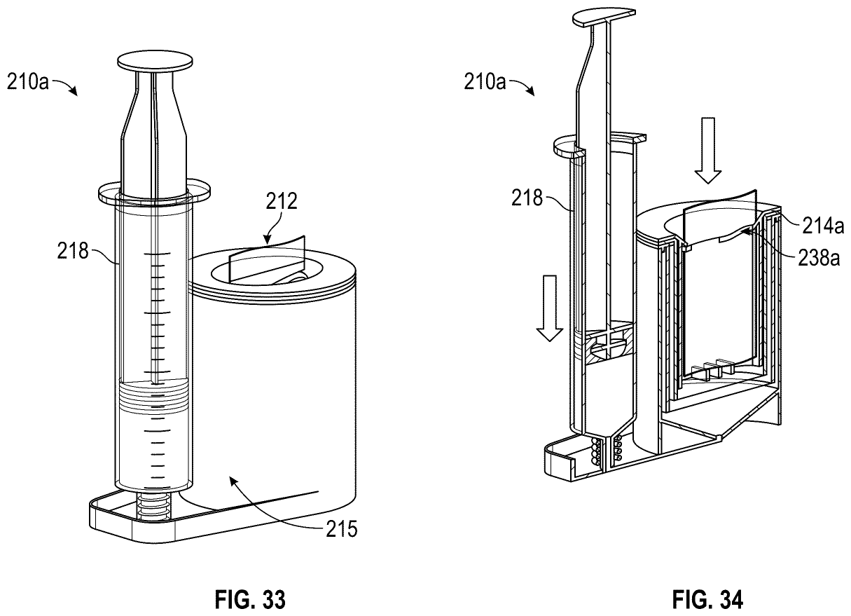

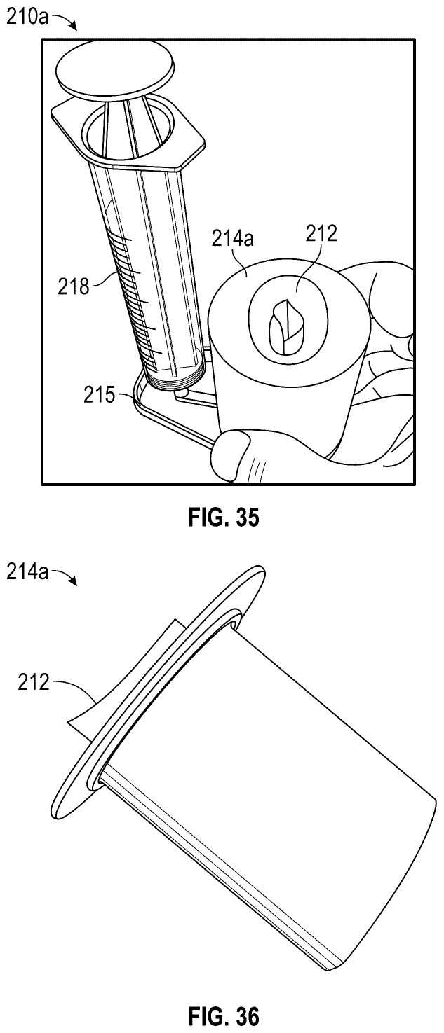

FIGS. 33 to 36 illustrate an alternative embodiment of a device 210a for recovering cells and cellular components in accordance with the present disclosure. The device 210a is the same as the device 210 but utilizes an alternative biopsy bag supporting device 214a with a biopsy bag attachment part 238a that is a slot which enables a biopsy bag 212 to be inserted from the top instead of being slid over the biopsy bag supporting device 214 from the bottom as with the biopsy bag attachment part 238 as shown in FIGS. 27 and 28.

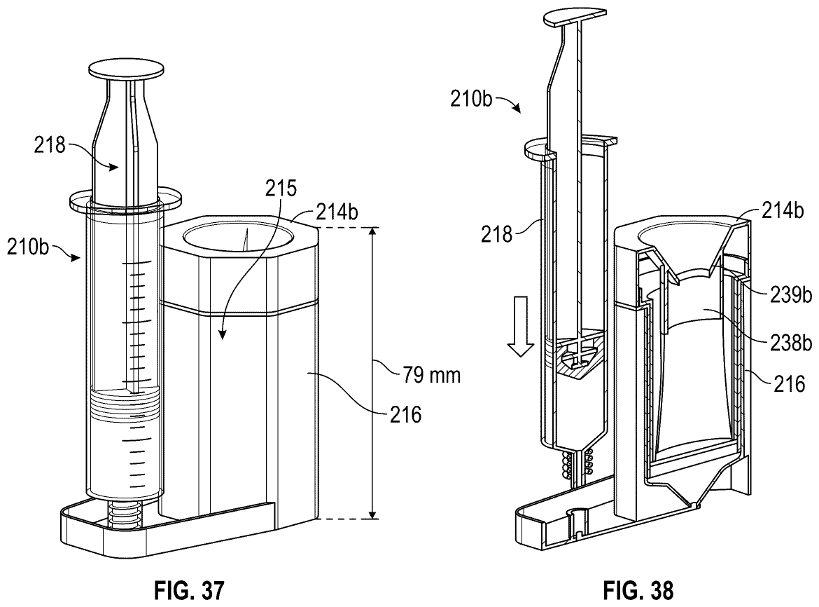

FIGS. 37 and 38 illustrate another alternative embodiment of a device 210b for recovering cells and cellular components in accordance with the present disclosure. The device 210b is the same as the device 210 but has an alternative biopsy bag supporting device 214b in which the biopsy bag attachment part 238 includes a downward protrusion that attaches to the hollow funnel 230b at a center point 239b, which creates a snugger fit for a biopsy bag 212 on the downward protrusion.

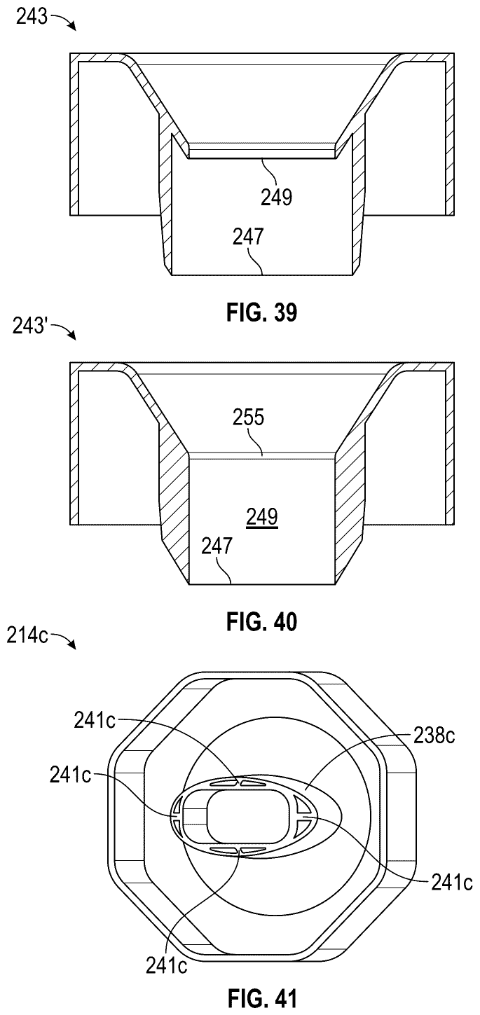

FIGS. 39 to 41 illustrate another alternative design for a biopsy bag supporting device 214c. FIGS. 39 and 40 illustrate different designs for a biopsy bag lead-in 243, 243′ which assists a user when sliding on a biopsy bag 212. FIG. 39 represents the basic configuration of the excisional biopsy (solid tissue specimen) intake portion of the lead-in 243. The lowermost or bottom portion 247 of the lead-in 243 is the width of the opening of a standard biopsy bag (FIG. 3). The biopsy bag fits over the bottom portion 247, as shown in FIG. 28. This configuration can be difficult for the user to fit the biopsy bag over the lower chute, but a narrower chute may result in the biopsy bag 212 slipping off once the user adds chunks of solid tissue into it. However, adding an inclined skirt to the bottom can make the part difficult to manufacture via injection molding, because of the engineering feature known as “undercut.” FIG. 40 therefore introduces a slanted “on-ramp skirt” design. The biopsy bag lead-in 243′ in FIG. 40 uses a combination of a slanted on-ramp skirt 247′ for the biopsy bag and increases the chute wall thickness so that there is no injection molding undercut 249 as seen in FIG. 39. That is, the lead-in 243′ has an inner wall 249 that extends downward from the inner corner 255 of the dispensing aperture with no undercut. This configuration resolves all three issues: (1) The biopsy bag 212 may be easily inserted over the chute because of the slanted “on ramp skirt”, (2) The tight fit of the biopsy bag 212 around the chute will help it to stay in place when the solid tissue chunks are added, and (3) The part may be easily injection-molded because of the absence of any undercut features. As an additional embodiment to make the part mold-friendly, FIG. 41 shows a design with open space that can be occupied by parts of an injection mold without changing the slanted skirt effect, while still avoiding creating any mold undercuts. FIG. 41 illustrates this biopsy bag supporting device 214c with an attachment part 238c with open space struts 241c consistent with the principals of injection moldability.

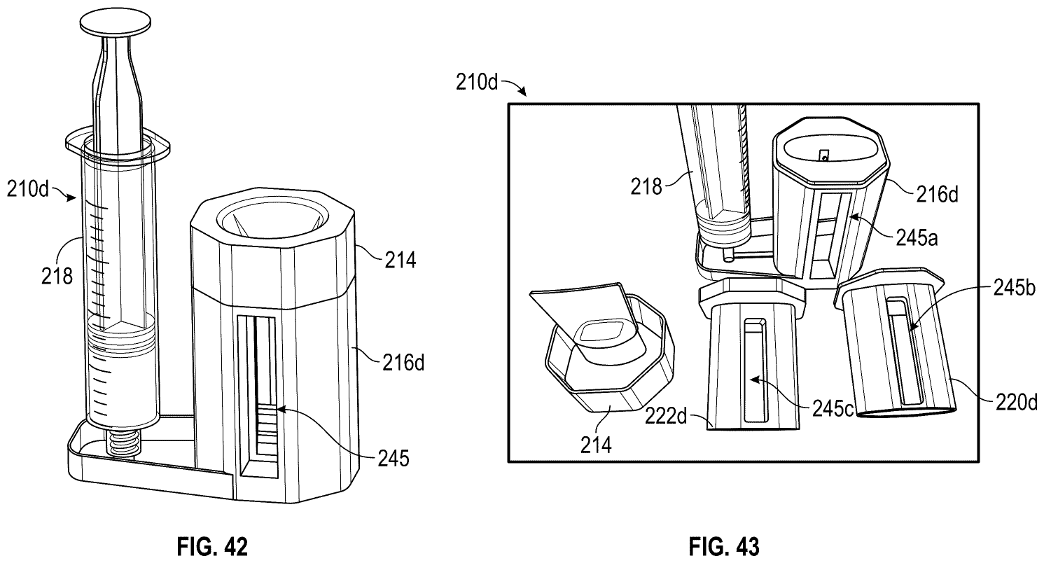

FIGS. 42 and 43 illustrate another alternative design for a device 210d for recovering cells and cellular components in accordance with the present disclosure. The device 210d includes one or more viewing windows 245 enabling a user to see the level of fluid and the levels of cells and debris that have accumulated inside the collection receptacles 218d, 220d, 222d. More specifically, the base receptacle 216d includes a first viewing window 245a, the lower sieve collection receptacle 220d includes a second viewing window 245b, and the upper sieve collection receptacle 222d includes a third viewing window 245c. When the parts are attached as shown in FIG. 42, the viewing windows 245a, 245b, 245c align so that the user can view through to the biopsy bag 212.

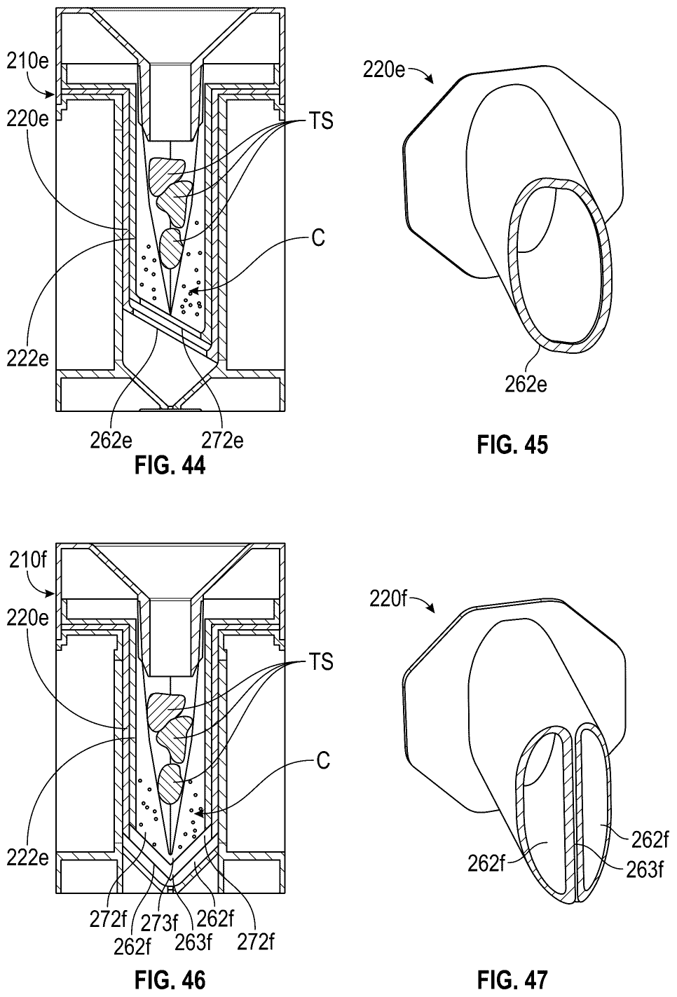

FIGS. 44 and 45 illustrate an alternative design for a lower sieve collection receptacle 220e and an upper sieve collection receptacle 222e. The lower sieve collection receptacle 220e has a sieve surface 262e that angles downwardly from one side to the other to assist in fluid flow. Likewise, the upper sieve collection receptacle 222e has a sieve surface 272e that angles downwardly from one side to the other to assist in fluid flow.

FIGS. 46 and 47 illustrate another alternative design for a lower sieve collection receptacle 220f and an upper sieve collection receptacle 222e. The lower sieve collection receptacle 220f has two sieve surfaces 262f that angle downwardly to meet at a center line 263f to assist in fluid flow. Likewise, the upper sieve collection receptacle 222e has two sieve surfaces 272e that angle downwardly to meet at a center line 273f to assist in fluid flow.

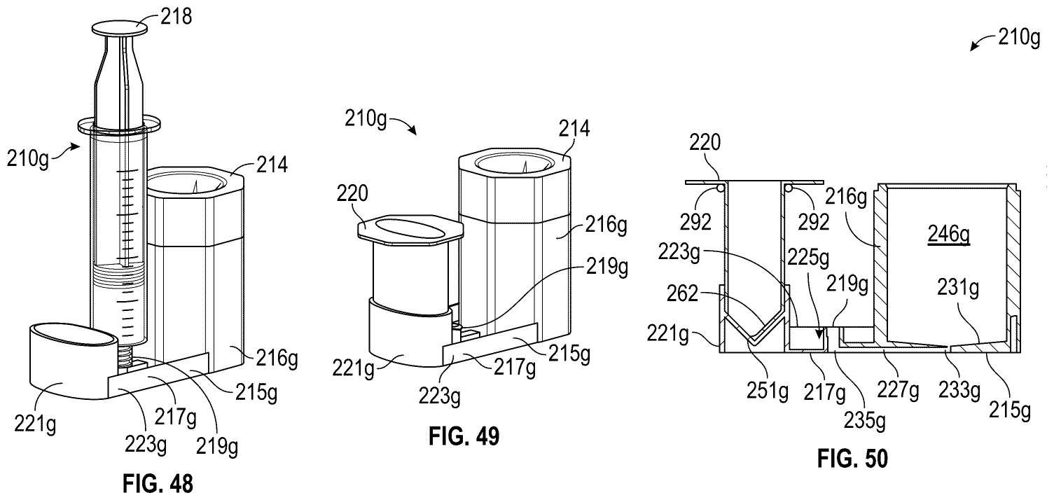

FIGS. 48 to 50 illustrate another alternative design for device 210g that utilizes an alternative base 215g. As with the previous embodiments, the base 215g is placed on a flat surface such as a countertop during use of the system 210g. In the illustrated embodiment, the base 215g includes a base receptacle 216g, a lower support 217g, an attachment port 219g, and a receptacle holder 221g. As seen in FIG. 50, the base receptacle 216g is located on one side of the lower support 217, and the receptacle holder 221g is located on the opposite side of the lower support 217 in the length direction, with the attachment port 219g located between the base receptacle 216g and the receptacle holder 221g. The lower support 217g further includes an outer wall 223g on the side of the attachment port 219g which extends from the base receptacle 216g to the receptacle holder 221g. The outer wall 223g creates a cavity 225g surrounding the attachment port 219g to catch fluid overflow at the attachment port 219g.

As seen in FIG. 50, the base 215g further includes a fluid channel 227g disposed within the lower support 217g. The fluid channel 227g places the inner space 246 of the base receptacle 216g in fluid communication with the inner space of the attachment port 219g. In the illustrated embodiment, the base receptacle 216g further includes a funnel portion 231g that funnels into a first end 233g of the fluid channel 227g. The opposite second end 235g of the fluid channel 227g is connected to and located below the attachment port 219g. Fluid within the inner space 246g of the base receptacle 216g thus flows down the funnel portion 231g, so that it can be sucked through the fluid channel 227g and into the fluid collection receptacle 218g via the attachment port 219g.

The receptacle holder 221g is configured to receive the lower sieve collection receptacle 220 after the lower sieve collection receptacle 220 has been removed from the base receptacle 216g, as seen in FIGS. 49 and 50. The receptacle holder 221g includes a receiving surface 251g that is generally sized and shaped to accept the lower sieve collection receptacle 220 so that the lower sieve collection receptacle 220 sits upright within the receptacle holder 221g. When sitting in the receptacle holder 237g, a user can collect cells from the lower sieve collection receptacle 220, for example, using a pipette. More specifically, the user can resuspend the cells in solution for extraction by a pipette. That is, the user can retrieve the cells that are caught on the sieve surface 262 and by adding a fluid (e.g. saline or buffer) to the lower sieve collection receptacle 220 while in the receptacle holder 221g to re-suspend the cells so that they can then be pipetted away for diagnostics.

As further seen in FIGS. 50 and 54, the lower sieve collection receptacle 220 and the upper sieve collection receptacle 222 can each include o-rings 292. The o-rings 292 are located under the respective upper edges of the lower sieve collection receptacle 220 and the upper sieve collection receptacle 222 and enable fluid tight seals when the lower sieve collection container 220 is placed in the base receptacle 216 and the upper sieve collection receptacle 222 is placed in the lower sieve collection receptacle 220.

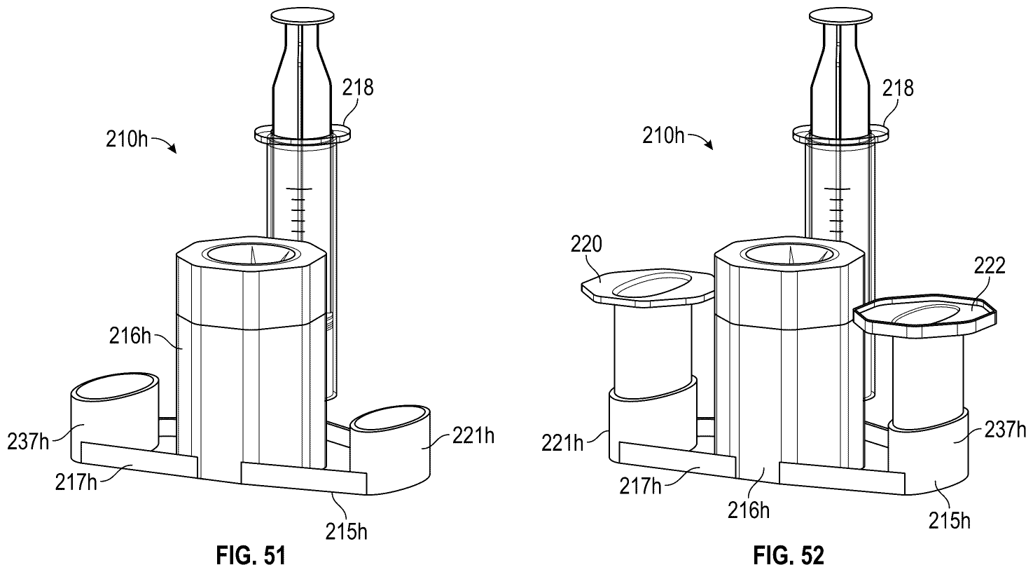

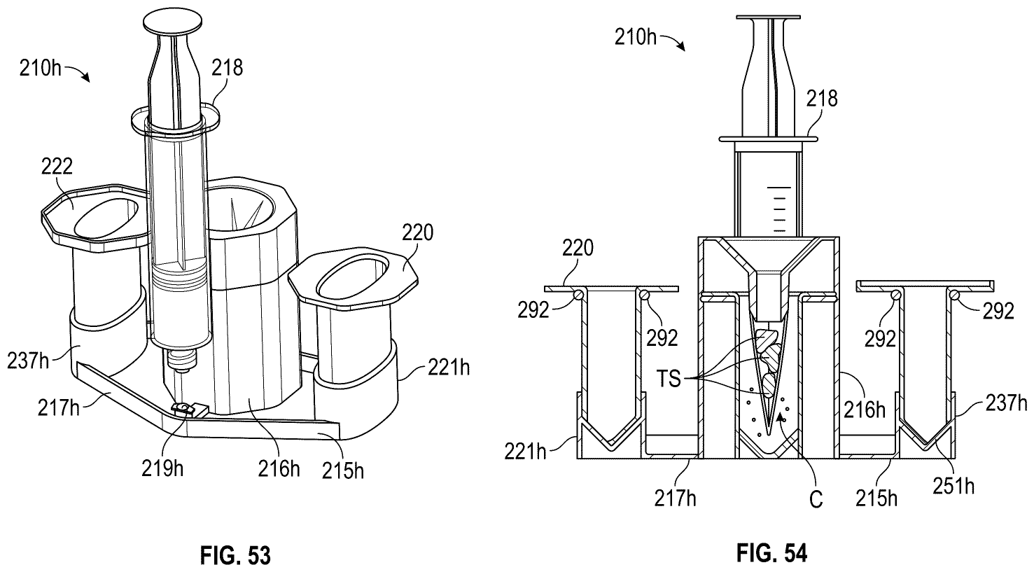

FIGS. 51 to 54 illustrate another alternative design for a device 210h that utilizes an alternative base 215h. As with the previous embodiments, the base 215h includes a base receptacle 216h, a lower support 217h, an attachment port 219h, and a first receptacle holder 221h for the lower sieve collection receptacle 220. The first receptacle holder 221h is generally the same as the receptacle holder 221g in FIGS. 48 to 50. The base 210h also includes a second receptacle holder 237h for the upper sieve collection receptacle 222.

The second receptacle holder 237h also generally functions the same way as the receptacle holder 221g in FIGS. 48 to 50. That is, the second receptacle holder 237h is configured to receive the upper sieve collection receptacle 222 after the upper sieve collection receptacle 222 has been removed from the base receptacle 216h, as seen in FIGS. 52 to 54. The second receptacle holder 237h includes a receiving surface 251h that is generally sized and shaped to accept the upper sieve collection receptacle 222 so that the upper sieve collection receptacle 222 sits upright within the second receptacle holder 237h. When sitting in the second receptacle holder 237h, a user can collect cells from the upper sieve collection receptacle 222, for example, using a pipette. More specifically, the user can resuspend the cells in solution for extraction by a pipette. That is, the user can retrieve the cells that are caught on the sieve surface 272 and by adding a fluid (e.g. saline or buffer) to the upper sieve collection receptacle 222 while in the second receptacle holder 237h to re-suspend the cells so that they can then be pipetted away for diagnostics.

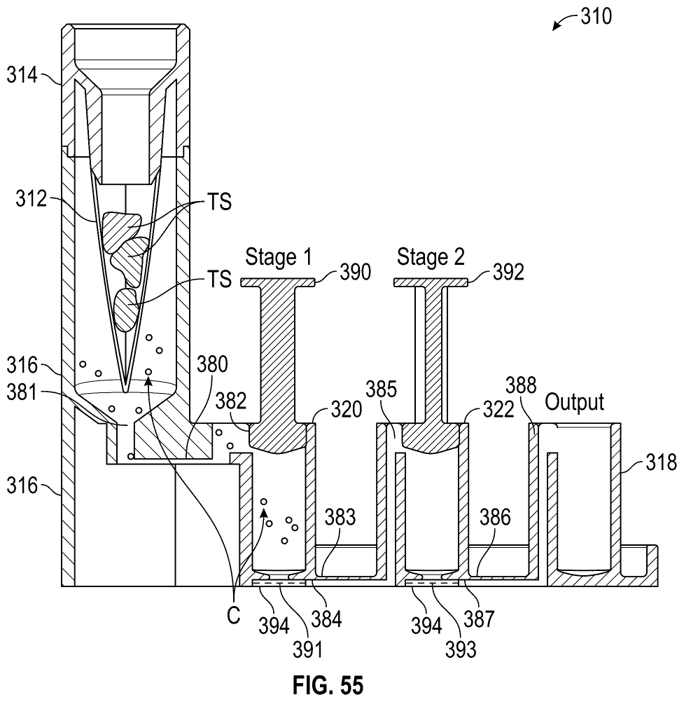

FIG. 55 illustrates another example embodiment for a device 310 for recovering cells and cellular components in accordance with the present disclosure. As with the previous embodiments, the device 310 is configured to separate fluid with the loose cells and cellular fragments into separate aliquots.

As with the previous embodiments, the device 310 includes one or more of a biopsy bag 312, a biopsy bag supporting device 314, a base receptacle 316, and a plurality of collection receptacles 318, 320, 322. In the illustrated embodiment, the plurality of collection receptacles 318, 320, 322 includes a fluid collection receptacle 318, a first sieve collection receptacle 320 and a second sieve collection receptacle 322. As with the previous embodiments, the plurality of collection receptacles 318, 320, 322 each include an inner space and are positioned and arranged to cause separation of fluid with the loose cells and cellular fragments into three separate aliquots A1, A2, A3 each in a collection receptacle 318, 320, 322.

As with the previous embodiments, the device 310 includes a biopsy bag supporting device 314. The biopsy bag supporting device 314 is configured to support the biopsy bag 312 at least partially within the base receptacle 316. The biopsy bag supporting device 314 can be generally sized and shaped and function in the same way as the biopsy bag supporting device(s) discussed above.

In the illustrated embodiment, the device 310 includes a base 315. The base 315 is placed on a flat surface such as a countertop during use of the system 310. In the illustrated embodiment, the base 315 includes the base receptacle 316 and the plurality of collection receptacles 318, 320, 322. The base receptacle 216 and the plurality of collection receptacles 318, 320, 322 are in fluid communication with each other. More specifically, the base 315 includes a first fluid channel 380 extending from a first end 381 to a second end 382, a second fluid channel 383 extending from a first end 384 to a second end 385, and a third fluid channel 386 extending from a first end 387 to a second end 388. The first fluid channel 380 places the base receptacle 316 in fluid communication with the first collection receptable 320. The second fluid channel 383 places the first sieve collection receptable 320 in fluid communication with the second sieve collection receptable 322. The third fluid channel 386 places the second sieve collection receptable 322 in fluid communication with the final fluid collection receptable 318.

In the illustrated embodiment, the base further includes a first plunger 390, a first filter 391, a second plunger 392 and a second filter 393. More specifically, the first sieve collection receptable 320 includes the first plunger 390 and the first filter 391, and the second sieve collection receptable 322 includes the second plunger 392 and the second filter 393. In the illustrated embodiment, the first sieve collection receptable 320 includes a first filter chamber 394, and the first filter 391 is located in the first filter chamber 394. The first filter 391 includes a sieve surface that fluid must flow through to leave the inner space of the first sieve collection receptable 320 and flow into the second fluid channel 383. The first filter chamber 394 is located at the bottom of the first sieve collection receptable 320 and at the first end 384 of the second fluid channel 383. Similarly, the second sieve collection receptable 322 includes a second filter chamber 395, and the second filter 393 is located in the second filter chamber 395. The second filter 393 includes a sieve surface that fluid must flow through to leave the inner space of the second sieve collection receptable 322 and flow into the third fluid channel 386. The second filter chamber 395 is located at the bottom of the second sieve collection receptable 322 and at the first end 387 of the third fluid channel 386. The first plunger 390 and the second plunger 392 are removable from the base 215. The first filter 391 has a larger pore size than the second filter 393. In the illustrated embodiment, the first filter 391 has a pore size of about 20 microns, with a range of 18-30 microns, and the second filter 393 has a pore size of about 7 microns, with a range of 3-10 microns. Those of ordinary skill in the art will recognize from this disclosure that the pore size can vary as needed or desired.

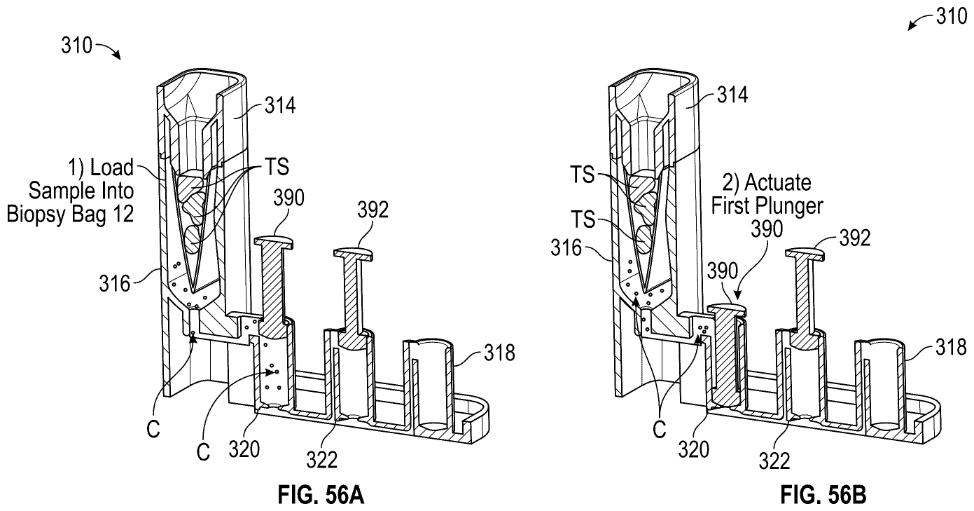

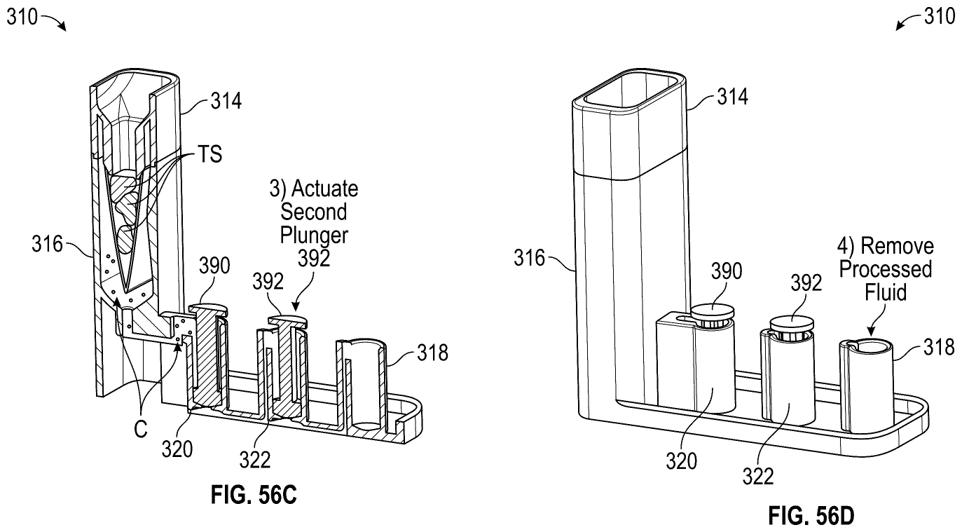

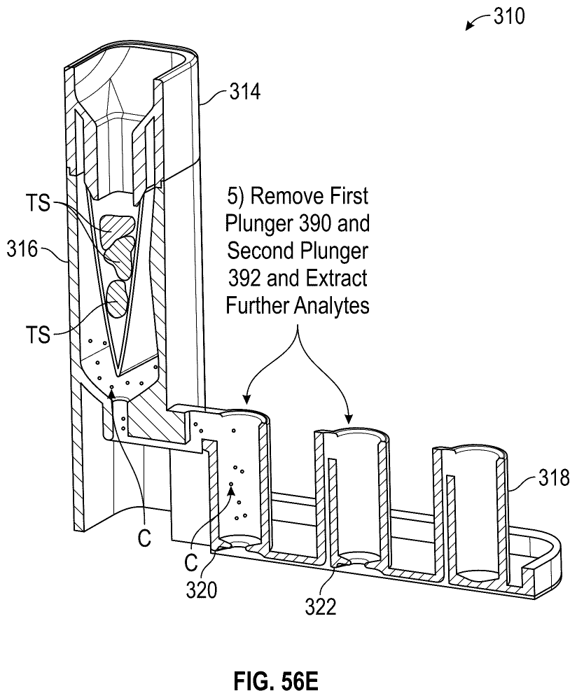

FIGS. 56A to 56E illustrate use of the device 310. Beginning with FIG. 56A, a user first places the biopsy bag supporting device 314 and attached biopsy bag 312 onto the base 215. The biopsy bag 312 contains a tissue specimen. The user then pours fluid (e.g., saline/buffer) into the biopsy bag supporting device 314. Preferably, the fluid should be poured so that it rises to a level that covers the tissue specimen in the biopsy bag 212. The fluid flows through the first fluid channel 380 and into the first sieve collection receptable 320 due to the force of gravity, carrying loose cells dislodged from the tissue specimen along with it, since the inner space of the first sieve collection receptable 320 is located below the base receptacle 316.

At FIG. 56B, the user actuates the first plunger 390. More specifically, the user pushes the first plunger 390 into the first sieve collection receptable 320, which causes the fluid to flow from the first sieve collection receptable 320, through the sieve surface of the first filter 391, through the second fluid channel 383, and into the second sieve collection receptable 322. The force of the first plunger 390 is needed since the second end 385 of the second fluid channel 383 which is the entry point to the second sieve collection receptable 322 is located above the first end 384 of the second fluid channel 383 and the force created by the user pushing on the plunger 390 is a more effective way to cause the fluid containing loose cells and analytes to be moved through filter 391 than relying on gravity or on suction from the distal end would be. As the fluid passes through the first filter 391, cellular material having a larger size than the pore size than the sieve surface of the first filter 391 remains in the inner space of the first sieve collection receptable 320 and cellular material having a smaller size than the pore size than the sieve surface of the first filter 391 moves into the inner space of the second sieve collection receptable 322.

At FIG. 56C, the user actuates the second plunger 392. More specifically, the user pushes the second plunger 392 into the second sieve collection receptable 322, which causes fluid to flow from the second sieve collection receptable 322, through the sieve surface of the second filter 393, through the third fluid channel 386, and into the final fluid collection receptable 318. The force of the second plunger 392 is needed since the second end 388 of the third fluid channel 386 which is the entry point to the fluid collection receptable 318 is located above the first end 387 of the third fluid channel 386, and the force created by the user pushing on the plunger 393 is a more effective way to cause the fluid containing loose cells and analytes to be moved through filter 393 than relying on gravity or on suction from the distal end would be. As the fluid passes through the second filter 393, cellular material having a larger size than the pore size than the sieve surface of the second filter 393 remains in the second sieve collection receptable 322 and cellular material having a smaller size than the pore size than the sieve surface of the second filter 393 moves into the inner space of the final fluid collection receptable 318.

At FIG. 56D, the user removes the fluid from the fluid collection receptable 318. For example, the user can pipette the fluid from the fluid collection receptable 318. This fluid contains exosomes, nucleic acids, proteins, and other molecular analytes that may be useful as biomarkers for cancer. These analytes are more relevant and predictive of a cancer patient's clinical course than any analytes recovered from peripheral blood draws because they come directly from the patient's tumor and are therefore present in higher concentration, and of higher concentration than any analytes recovered in urine because they originate from the freshly cut surfaces of the tumor tissue.

At FIG. 56E, the user removes the first plunger 390 from the first sieve collection receptable 320 and the second plunger 392 from the second sieve collection receptable 322. The user the removes the fluid and cellular material from the first sieve collection receptable 320 and the second sieve collection receptable 322. For example, the user can pipette the fluid from the first sieve collection receptable 320 and the second sieve collection receptable 322. The fluid from the first sieve collection receptable 320 contains dislodged tumor cells which originated with the cut tissue of the patient's solid tumor, and therefore may have higher diagnostic and treatment predictive value than cells recovered from peripheral blood draws, or from urine, which may have undergone molecular changes after leaving the tumor. The fluid from the second sieve collection receptable 322 contains cellular debris, cell fragments, red blood cells, and single tumor cells, which may be useful for single cell analysis, organoid growth, or patient-specific tumor model creation. In an embodiment, the user can add fluid to the first sieve collection receptable 320 and the second sieve collection receptable 322 to resuspend the cellular material for pipetting.

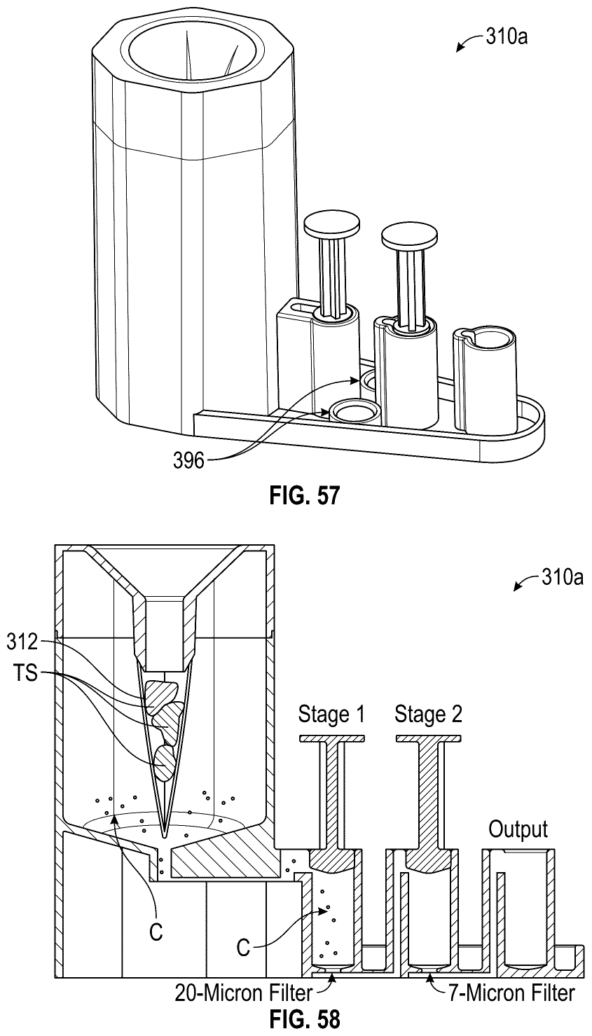

FIGS. 57 and 58 illustrate an alternative design of a device 310a, which is similar to the device 310 and further includes two plunger cups 396 configured to hold the first plunger 390 and the second plunger 392 when they are removed from the first sieve collection receptable 320 and the second sieve collection receptable 322. This enables the user to reuse the device 310a later and not risk misplacing the plungers 390, 392. The device 310a further has larger dimensions, with the pore size of the first filter being about 20 microns and the pore size of the second filter being about 7 microns.

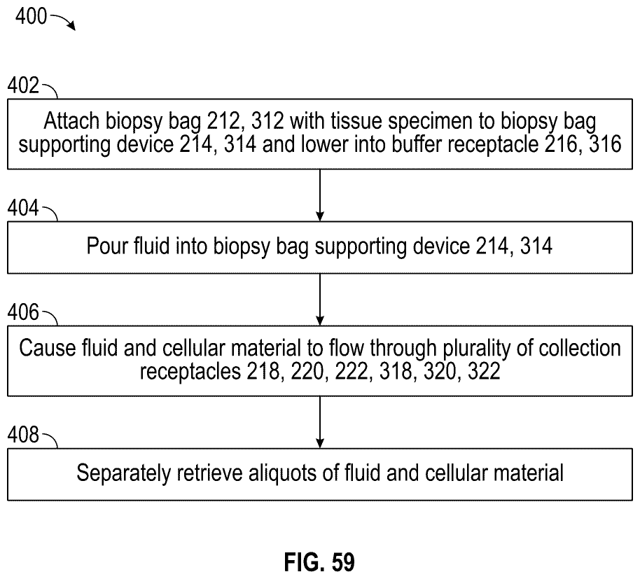

FIG. 59 illustrates an example embodiment of a method 400 of recovering cells and cellular components in accordance with the present disclosure. In an embodiment, the method 400 is a method of using one of the devices 10, 210, 310 and/or the alternative embodiments described herein to recover cells and cellular components. Those or ordinary skill in the art will recognize from this disclosure, however, that the method 400 can also be practiced with alternative systems and does not necessary require the devices 10, 210, 310 shown. Those of ordinary skill in the art will also recognize from this disclosure that certain steps of the method 400 can be added, removed or altered without departing from the spirit and scope of the present disclosure.

At step 402, when a technician in a clinical pathology lab receives a tissue specimen TS (e.g., a specimen of resected bladder tumor), he or she places it in a biopsy bag 12, 212, 312 as usual, but instead of placing the full biopsy bag 12, 212, 312 in a cassette and into a processor box, the biopsy bag 12, 212, 312 is attached to a biopsy bag supporting device 14, 214, 314 as described herein. The assembly of the biopsy bag supporting device 14, 214, 314 and the biopsy bag 12, 212, 312 containing the tissue are lowered into the base receptacle 16, 216, 316, as shown for example in FIGS. 22 and 55. Alternatively, the biopsy bag 12, 212, 312 can be placed into the biopsy bag supporting device 14, 214, 314 from above, as shown for example in FIG. 34.

At step 404, the technician or other individual pours fluid into the biopsy bag supporting device 14, 214, 314. Here, the fluid includes saline or buffer solution, such as phosphate buffered saline (PBS) solution, but the method 400 can also work with other types of fluid. For example, the fluid can be poured into the hollow funnel 230 as described herein, so that it flows through the dispensing aperture 232 and into the biopsy bag 12, 212, 314, and then through the biopsy bag pores. Preferably, the fluid should be poured so that it rises to a level that covers the tissue specimen in the biopsy bag 12, 212, 312. In an embodiment, step 404 can be performed without or with an alternative type of biopsy bag supporting device 214, 314, for example, by suspending the biopsy bag 12, 212, 312 at least partially within the base receptacle 16, 216, 316 in another manner while pouring the fluid into the biopsy bag 212, 314.

At step 406, the technician or other individual causes the fluid and cellular material to flow through the plurality of collection receptacles 18, 20, 22, 218, 220, 222, 318, 320, 322 as described herein. As discussed above, there are various ways to cause the fluid and cellular material to flow through the plurality of collection receptacles 18, 20, 22, 218, 220, 222, 318, 320, 322.

In an embodiment, the technician applies mild agitation and/or warm incubation to encourage loose cells C to release exosomes and to disperse through the holes of the biopsy bag 12, 212, 312 into the fluid. In an embodiment, the mild agitation and/or warm incubation can be performed for approximately five minutes. This creates a fluid which combines with the loose cells and cellular fragments.

With the device 210, some loose cells and cellular fragments will flow with the fluid into the base receptacle 216, the lower sieve collection receptacle 220, and/or the upper sieve collection receptacle 222 even before any mild agitation and/or warm incubation due to the relative positioning and surfaces of the components. The technician also draws the fluid into the fluid collection receptacle 218 by actuating the plunger 251. If the fluid collection receptacle 218 is not already attached to the base 215, the fluid collection receptacle 218 is attached to the base 215 by placing the tip 253 into the attachment port 219, as seen for example in FIGS. 22 and 23. The user then causes the fluid to be suctioned from the inner space 246 of the base receptacle 216, through the fluid channel 227, and into the inner space 252 of the fluid collection receptacle 218. In the illustrated embodiment, the user causes the suction by withdrawing the piston or plunger 251 while the fluid collection receptacle 218 is attached to the attachment port 219.

With the device 310, step 406 includes pressing the plungers 390, 392 as discussed above. That is, the technician presses the first plunger 390 to cause the fluid and cellular material to flow from the first sieve collection receptable 320, through the sieve surface of the first filter 391, through the second fluid channel 383, and into the second sieve collection receptable 322. The technician then presses the second plunger 392 to cause the fluid and cellular material to flow from the second sieve collection receptable 322, through the sieve surface of the second filter 393, through the third fluid channel 386, and into the final fluid collection receptable 318. Those of ordinary skill in the art will further recognize from this disclosure that there are also other ways to cause fluid to flow as desired besides the methods described herein.

After step 406, the final fluid collection receptacle 18, 218, 318, the first or lower sieve collection receptacle 20, 220, 320, and the second or upper sieve collection receptacle 22, 222, 322 should contain different aliquots A1, A2, A3. With the device 210, this is the result of the fluid flowing into the upper sieve collection receptacle 222, through the sieve surface 272 of the upper sieve collection receptacle 222 and the sieve surface 262 of the lower sieve collection receptacle 220, and into the fluid collection receptacle 218. This is also the result of the fluid being suctioned through the fluid channel 227 into the fluid collection receptacle 218. With the device 310, this is the result of the fluid flowing into the first sieve collection receptacle 320, through the sieve surface of the first filter 391, into the second sieve collection receptacle 322, through the sieve surface of the second filter 393, and into the final fluid collection receptacle 218, 318.

In an embodiment, the collection receptable with the larger pore size (here, the upper sieve collection receptacle 222 of the device 210 or the first sieve collection receptacle 320 of the device 310) collects dislodged or friable cells (e.g., the first aliquot A1) that have fallen off from the solid (“parent”) tissue specimen TS. These include in-tact cells that can be used, for example, for metabolomic studies (via extraction of metabolites with 80% methanol), genomics studies. organoid growth, and/or nucleic acid (DNA and RNA) analysis. RNA analysis is sometimes also called transcriptomics.

In an embodiment, the collection receptable with the smaller pore size (here, the lower sieve collection receptacle 220 of the device 210 or the second sieve collection receptacle 322 of the device 310) collects cellular debris and some single cells (e.g., the second aliquot A2), and separates it from the final, valuable fluid (e.g., the third aliquot A3) that is collected by the final fluid collection receptacle 218, 318. The second aliquot A2 includes cell fragments, red blood cells, debris, and some single cells. The cellular debris currently has no clinical value and can be discarded, but the single cells present in A2 may be used for single cell analysis, organoid growth, or for the creation of patient-specific tumor models. Those of ordinary skill in the art should recognize that new uses may arise for the second aliquot A2 in the future.

The final fluid collection receptacle 218, 318 collects the final, valuable fluid (e.g., the third aliquot A3). With the device 210, the third aliquot A3 includes the fluid or material that passes through the sieve surface 272 of the upper sieve collection receptacle 222 and the sieve surface 262 of the lower sieve collection receptacle 220. That fluid contains exosomes and/or the contents of exosomes, such as nucleic acids, proteins, and other analytes that could be used as cancer biomarkers. With the device 310, the third aliquot A3 includes the fluid or material that passes through the first filter 391 and the second filter 393.

At step 408, the technician or other individual can separately retrieve the aliquots of fluid and cellular material of interest as described herein. In different embodiments, the technician or other individual can remove respective containers and pour out the fluid and cellular material or pipette out the fluid and cellular material as described herein.

The technician or other individual can withdraw the first aliquot A1 from the sieve collection receptacle having the sieve surface with the larger pore size (e.g., the upper sieve collection receptacle 222 of the device 210 or the first sieve collection receptacle 320 of the device 310). For example, with the device 210, the technician removes the upper sieve collection receptacle 222 from the fluid collection receptacle 218 and the lower sieve collection receptacle 220. This allows the user to use the recovered in-tact cells, for example, for metabolomics (via extraction of metabolites with 80% methanol), organoid growth, and nucleic acid (DNA and RNA) analysis. In another example, with the device 310, the technician can pipette the fluid from the second sieve collection receptacle 322. In an embodiment, the technician can retrieve first aliquot A1 by adding a fluid (e.g. saline or buffer) to re-suspend the cells so that they can then be pipetted away, for example as discussed above.

In an embodiment, the technician or other individual can withdraw the second aliquot A2 from the sieve collection receptacle having the sieve surface with the smaller pore size (e.g., the lower sieve collection receptacle 220 of the device 210 or the second sieve collection receptacle 322 of the device 310). For example, with the device 210, the technician removes the lower sieve collection receptacle 220 from the fluid collection receptacle 218. In an embodiment, the lower sieve collection receptacle 220 contents (cell fragments, red blood cells and debris) can be discarded, or kept to retrieve single cells that may be present in it, which could be useful for single cell analysis. The general purpose of the lower sieve collection receptacle 220 is to remove the cell fragments, red blood cells and debris from the fluid that flows into the fluid collection receptacle 218, which contains exosomes and/or the contents of exosomes such as nucleic acids, proteins, and other analytes that could be used as cancer biomarkers, and can further be used for investigational research studies to correlate exosomes and their constituent contents such as nucleic acids, proteins, and other biomarkers associated with certain tumors. In another example, with the device 310, the technician can pipette the fluid from the first sieve collection receptacle 320. In an embodiment, the technician can retrieve second aliquot A2 by adding a fluid (e.g. saline or buffer) to re-suspend the cells so that they can then be pipetted away, for example as discussed above.

In an embodiment, the technician or other can individual withdraw the third aliquot A3 from the residual fluid collection receptacle 18, 218, 318. For example, with the device 210, the technician can remove the fluid collection receptacle 218 from the base 215 and then withdraw the fluid from the fluid collection receptacle 218. In another example, with the device 310, the technician can pipette the fluid from the fluid collection receptacle 318. The third aliquot A3 generally includes exosomes, nucleic acids, proteins, and other analytes and can further be used for investigational research studies to correlate exosomes, expressed proteins, and other biomarkers associated with certain tumors. The contents of this fluid is far more specific to the tumor than exosome-containing fluids from the body, such as urine or peripheral blood, and is therefore an excellent substrate for tumor biomarker analysis.

The steps of the methods 100, 400 discussed herein enable collection of cells normally lost to contamination, as well as the fluid that bathed these cells which provides a resource of previously discarded diagnostic cells and fluid with exosomes. This recovered material can be frozen and then sent to clinicians and scientists with expertise in their respective fields for the evaluation of exosomes, metabolites and nucleic acids, respectively. Aliquots of recovered material for other uses, such as organoids, proteomics, metabolomics, and nucleic acids are valuable outputs of the methods 100, 400 and/or the systems 10, 210, 310 disclosed herein.

Because all the steps of performing the methods 100, 400 and/or using the systems 10, 210, 310 occur before exposure to formalin, and the separation process is quick (less than 10 minutes), the analytes collected and recovered will retain their integrity. Recovered nucleic acids (DNA and RNA), for example, are expected to remain long-chain, not fragmented, and not crosslinked, and hence of the highest diagnostic value for multi-omic testing.

An advantage of the disclosed systems 10, 210, 310 and/or methods 100, 400 disclosed herein is that clinicians treating patients with solid tumors will have the benefit of additional diagnostic information that can inform treatment decisions, including whether a patient must be subjected to radiation therapy and chemotherapy, or be spared from the serious side effects of those treatments and instead be able to enter a clinical trial for a precision medicine cancer therapeutic.

Another advantage of the disclosed systems 10, 210, 310 and/or methods 100, 400 disclosed herein is that by “washing” away the loose cells and cellular debris from solid tumor excised tissue and tumor fragments, once the tissue in the plastic cassette is processed in the clinical pathology laboratory, fewer “floaters” will end up mixing with another patient's sample in a processing box. This may reduce the incidence of “false positives”, for example, thinking that there is a neoplastic tumor in another specimen, but actually a “floater” that came from another patient's tissue sample.

Yet another advantage of the disclosed systems 10, 210, 310 and/or methods 100, 400 disclosed herein is that the biomarker analytes (exosomes, proteins, nucleic acids) that the use of the disclosed systems 10, 210, 310 and/or methods 100, 400 yields are likely to be more clinically relevant than similar biomarkers obtained through routine urine specimens, because the fluid collected in the present invention was in direct contact with the tumor tissue. In urine, exosomes may be present that originated from all parts of a patient's body. By contrast, the investigational diagnostic or research benefit of the liquid specimen produced by the present invention is that the recovered exosomes have originated only from the urinary bladder tumor cells.

In an embodiment, the parts disclosed herein can be injection molded from plastic. Those of ordinary skill in the art will also recognize from this disclosure that other manufacturing methods can be used.

It should be understood that various changes and modifications to the apparatuses and methods described herein will be apparent to those skilled in the art and can be made without diminishing the intended advantages.

### General Interpretation of Terms

In understanding the scope of the present invention, the term “comprising” and its derivatives, as used herein, are intended to be open-ended terms that specify the presence of the stated features, elements, components, groups, and/or steps, but do not exclude the presence of other unstated features, elements, components, groups, integers and/or steps. The foregoing also applies to words having similar meanings such as the terms, “including”, “having” and their derivatives. Also, the terms “part,” “section,” or “element” when used in the singular can have the dual meaning of a single part or a plurality of parts.

The term “configured” as used herein to describe a component, section or part of a device includes hardware that is constructed to carry out the desired function.

The terms “first,” “second,” “third,” etc. as used herein are to distinguish like parts, points, locations, etc. and can be reordered or used interchangeably. The terms “first,” “second,” “third,” etc. are not intended to be limiting. For example, the lower sieve collection receptable is described above as a “first” sieve collection receptable and the upper sieve collection receptable is described above as a “second” sieve collection receptable, but the upper sieve collection receptable can also be considered a “first” sieve collection receptable and the lower sieve collection receptable a “second” sieve collection receptable.

While only selected embodiments have been chosen to illustrate the present invention, it will be apparent to those skilled in the art from this disclosure that various changes and modifications can be made herein without departing from the scope of the invention as defined in the appended claims. For example, the size, shape, location or orientation of the various components can be changed as needed and/or desired. Components that are shown directly connected or contacting each other can have intermediate structures disposed between them. The functions of one element can be performed by two, and vice versa. The structures and functions of one embodiment can be adopted in another embodiment. It is not necessary for all advantages to be present in a particular embodiment at the same time. Every feature which is unique from the prior art, alone or in combination with other features, also should be considered a separate description of further inventions by the applicant, including the structural and/or functional concepts embodied by such features. Thus, the foregoing descriptions of the embodiments according to the present invention are provided for illustration only, and not for the purpose of limiting the invention as defined by the appended claims and their equivalents.

What is claimed is:

1. A device for recovering cells and cellular components, the device comprising:
a plurality of collection receptacles positioned and arranged with respect to each other so as to separate fluid with the loose cells and cellular fragments into separate aliquots,
the plurality of collection receptacles being separable from each other after the fluid with the loose cells and cellular fragments has been separated into the separate aliquots.

2. The device of claim 1, wherein
the plurality of collection receptacles includes at least a sieve collection receptacle which retains an aliquot including cells dislodged from harvested tissue, such as friable cells from a tumor mass that has been biopsied, and a fluid collection receptacle which retains another aliquot including fluid containing exosomes or the contents of exosomes, such as nucleic acids, proteins, and other analytes that could be used as cancer biomarkers.

3. The device of claim 1, wherein
the plurality of collection receptacles includes at least a first sieve collection receptacle which retains a first aliquot including friable cells, a second sieve collection receptacle which retains a second aliquot including cell fragments, red blood cells or other debris, and a fluid collection receptacle which retains a third aliquot including fluid containing exosomes or the contents of exosomes.

4. The device of claim 1, wherein
the plurality of collection receptacles includes an upper sieve collection receptacle and a lower sieve collection receptacle,
the upper sieve collection receptacle includes a first outer surface, a first inner space within the first outer surface and a first sieve surface,
the lower sieve collection receptacle includes a second outer surface, a second inner space within the second outer surface, and a second sieve surface having a smaller pore size than the first sieve surface, and
the upper sieve collection receptacle is positioned at least partially within the second inner space of the lower sieve collection receptacle when the fluid with the loose cells and cellular fragments is separated.

5. The device of claim 4, wherein
the plurality of collection receptacles are positioned and arranged with respect to each other such that pouring the fluid with the loose cells and cellular fragments into the first inner space of the upper sieve collection receptacle causes at least some of the fluid to flow through the first sieve surface of the upper sieve collection receptacle and into the second inner space of the lower sieve collection receptacle, then through the second sieve surface of the lower sieve collection receptacle and into a base receptacle.

6. The device of claim 1, comprising
a biopsy bag supporting device configured to support a biopsy bag containing a tissue specimen at least partially within at least one of the plurality of collection receptacles.

7. The device of claim 1, comprising
the plurality of collection receptacles includes, a base receptacle, a fluid collection receptacle, an upper sieve collection receptacle and a lower sieve collection receptacle,
the base receptacle is configured to at least partially receive at least one of the upper sieve collection receptacle and the lower sieve collection receptacle,
the fluid collection receptacle is fluidly connected to the base receptacle via a fluid channel so that fluid can be suctioned into the fluid collection receptacle from the base receptacle via the fluid channel.

8. A method for recovering cells and cellular components, the method comprising:
placing a biopsy bag with a tissue specimen at least partially within a supporting device;
causing fluid that has been mixed with the tissue specimen to flow through a first sieve collection receptacle, through a second sieve collection receptacle, and into a fluid collection receptacle such that the fluid with loose cells and cellular fragments from the tissue sample separates into separate aliquots; and
retrieving the separate aliquots from at least two of the first sieve collection receptacle, the second sieve collection receptacle and the fluid collection receptacle for further diagnostics.

9. The method of claim 8, comprising
pouring the fluid into the biopsy bag such that at least some of the fluid flows through a first sieve surface of the first sieve collection receptacle and through a second sieve surface of the second sieve collection receptacle.

10. The method of claim 8, comprising
causing the fluid to flow into the fluid collection receptacle includes suctioning the fluid into the fluid collection receptacle to create the separate aliquot in the fluid collection receptacle.

11. The method of claim 8, wherein
causing the fluid to flow into the fluid collection receptacle includes separating the fluid and any contents therein into a first aliquot, a second aliquot and a third aliquot,
the first aliquot includes cells dislodged from harvested tissue, such as friable cells from a tumor mass that has been biopsied, retained within the first sieve collection receptacle,
the second aliquot includes cell fragments, red blood cells or other debris, and some single cells retained within the second sieve collection receptacle, and
the third aliquot includes fluid containing exosomes or the contents of exosomes, such as nucleic acids, proteins, and other analytes that could be used as cancer biomarkers retained within the fluid collection receptacle.

12. The method of claim 11, comprising
separating at least one of the first sieve collection receptacle, the second sieve collection receptacle, and the fluid collection receptacle from the at least another of the first sieve collection receptacle, the second sieve collection receptacle, and the fluid collection receptacle.

13. The method of claim 8, comprising
retaining friable cells within the at least one of the first sieve collection receptacle, the second sieve collection receptacle and the fluid collection receptacle, and
retaining fluid containing exosomes or the contents of exosomes within the at least another of the first sieve collection receptacle, the second sieve collection receptacle and the fluid collection receptacle.

14. The method of claim 8, comprising
causing the fluid to flow into the fluid collection receptacle includes actuating one or more pistons.

15. A device for recovering cells and cellular components, the device comprising:
a first sieve collection receptacle including a first inner space and a first sieve surface; and
a second sieve collection receptacle including a second inner space and a second sieve surface having a smaller pore size than the first sieve surface; and
a fluid collection receptacle including a third inner space,
the first sieve collection receptacle, the second sieve collection receptacle, and the fluid collection receptacle being positioned and arranged such that fluid containing loose cells and cellular fragments can be separated with the first sieve collection receptacle containing cells dislodged from harvested tissue, such as friable cells from a tumor mass that has been biopsied, from the fluid within the first inner space, the lower sieve collection receptacle containing cellular debris or single cells from the fluid that has passed through the first sieve surface and into the second inner space, and the fluid collection receptacle containing fluid and exosomes, nucleic acids, proteins or other analytes that could be useful as cancer biomarkers that has passed through the first sieve surface and the second sieve surface and into the third inner space.

16. The device of claim 15, further comprising
a base receptacle configured to at least partially receive at least one of the first sieve collection receptacle and the second sieve collection receptacle.

17. The device of claim 15, further comprising
a biopsy bag supporting device configured to support the biopsy bag containing the tissue specimen as the fluid separates between the first sieve collection receptacle, the second sieve collection receptacle, and the fluid collection receptacle.

18. The device of claim 15, wherein
the fluid collection receptacle is a syringe, and
applying a suction force with the syringe causes the remaining fluid to flow into the syringe.

19. The device of claim 15, wherein
the lower sieve collection receptacle and the upper sieve collection receptacle are positioned and arranged such that when fluid containing loose cells and cellular fragments is poured into the third inner space of the upper sieve collection receptacle, the fluid flows through the second sieve surface and into the second inner space of the lower sieve collection receptacle.

20. The device of claim 15, comprising
one or more pistons that when actuated cause the fluid to flow through the first sieve surface and the second sieve surface and separate between the first sieve collection receptacle, the second sieve collection receptacle, and the fluid collection receptacle.
